(19) United States
(12) Patent Application Publication
(10) Pub. No.: US 2009/0326019 A1
(43) Pub. Date: Dec. 31, 2009

(54) 3,4-BICYCLIC PYRROLIDINE ANTIVIRALS

(76) Inventors: Yat Sun Or, Watertown, MA (US); Jiang Long, Wayland, MA (US); Ce Wang, Waltham, MA (US); Lu Ying, Belmont, MA (US); Yao-Ling Qiu, Andover, MA (US)

Correspondence Address: ELMORE PATENT LAW GROUP, PC, 515 Groton Road, Unit 1R, Westford, MA, 01886, US

(21) Appl. No.: 12/482,804

(22) Filed: Jun. 11, 2009

Related U.S. Application Data
(60) Provisional application No. 61/060,705, filed on Jun. 11, 2008.

(51) Int. Cl. A61K 31/427; C07D 417/04; C12N 7/06; A61P 31/12

(52) U.S. Cl. 514/365; 548/181; 435/238

(57) ABSTRACT

The present invention discloses compounds of formula (I), or pharmaceutically acceptable salts, esters, or prodrugs thereof:

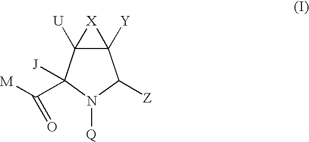

which inhibit RNA-containing virus, particularly the hepatitis C virus (HCV). Consequently, the compounds of the present invention interfere with the life cycle of the hepatitis C virus and are also useful as antiviral agents. The present invention further relates to pharmaceutical compositions comprising the aforementioned compounds for administration to a subject suffering from HCV infection. The invention also relates to methods of treating an HCV infection in a subject by administering a pharmaceutical composition comprising the compounds of the present invention. The present invention relates to novel antiviral compounds represented herein above, pharmaceutical compositions comprising such compounds, and methods for the treatment or prophylaxis of viral (particularly HCV) infection in a subject in need of such therapy with said compounds.

### RELATED APPLICATION

This application claims the benefit of U.S. Provisional Application No. 61/060,705, filed on Jun. 11, 2008. The entire teachings of the above application are incorporated herein by reference.

### TECHNICAL FIELD

The present invention relates to novel anti-infective agents. Specifically, the present invention relates to compounds, compositions, a method for inhibiting hepatitis C virus (HCV) polymerase, a method for inhibiting HCV viral replication, and a method for treating or preventing HCV infection. The invention further includes process by which to make the compounds of the present invention.

### BACKGROUND OF THE INVENTION

Infection with HCV is a major cause of human liver disease throughout the world. In the US, an estimated 4.5 million Americans are chronically infected with HCV. Although only 30% of acute infections are symptomatic, greater than 85% of infected individuals develop chronic, persistent infection. Treatment costs for HCV infection have been estimated at $5.46 billion for the US in 1997. Worldwide over 200 million people are estimated to be infected chronically. HCV infection is responsible for 40-60% of all chronic liver disease and 30% of all liver transplants. Chronic HCV infection accounts for 30% of all cirrhosis, end-stage liver disease, and liver cancer in the U.S. The CDC estimates that the number of deaths due to HCV will minimally increase to 38,000/year by the year 2010.

Due to the high degree of variability in the viral surface antigens, existence of multiple viral genotypes, and demonstrated specificity of immunity, the development of a successful vaccine in the near future is unlikely. Alpha-interferon (alone or in combination with ribavirin) has been widely used since its approval for treatment of chronic HCV infection. However, adverse side effects are commonly associated with this treatment: flu-like symptoms, leukopenia, thrombocytopenia, depression from interferon, as well as anemia induced by ribavirin (Lindsay, K. L. (1997) Hepatology 26 (suppl 1): 71S-77S). This therapy remains less effective against infections caused by HCV genotype 1 (which constitutes ˜75% of all HCV infections in the developed markets) compared to infections caused by the other 5 major HCV genotypes. Unfortunately, only ˜50-80% of the patients respond to this treatment (measured by a reduction in serum HCV RNA levels and normalization of liver enzymes) and, of responders, 50-70% relapse within 6 months of cessation of treatment. Recently, with the introduction of pegylated interferon (Peg-IFN), both initial and sustained response rates have improved substantially, and combination treatment of Peg-IFN with ribavirin constitutes the gold standard for therapy. However, the side effects associated with combination therapy and the impaired response in patients with genotype 1 present opportunities for improvement in the management of this disease.

First identified by molecular cloning in 1989 (Choo, Q-L et al (1989) Science 244:359-362), HCV is now widely accepted as the most common causative agent of post-transfusion non-A, non-B hepatitis (NANBH) (Kuo, G et al (1989) Science 244:362-364). Due to its genome structure and sequence homology, this virus was assigned as a new genus in the Flaviviridae family. Like the other members of the Flaviviridae, such as flaviviruses (e.g. yellow fever virus and Dengue virus types 1-4) and pestiviruses (e.g. bovine viral diarrhea virus, border disease virus, and classic swine fever virus) (Choo, Q-L et al (1989) Science 244:359-362; Miller, R. H. and R. H. Purcell (1990) Proc. Natl. Acad. Sci. USA 87:2057-2061), HCV is an enveloped virus containing a single strand RNA molecule of positive polarity. The HCV genome is approximately 9.6 kilobases (kb) with a long, highly conserved, noncapped 5′ nontranslated region (NTR) of approximately 340 bases which functions as an internal ribosome entry site (IRES) (Wang C Y et al ‘An RNA pseudoknot is an essential structural element of the internal ribosome entry site located within the hepatitis C virus 5′ noncoding region’ RNA—A Publication of the RNA Society. 1(5): 526-537, 1995 July). This element is followed by a region which encodes a single long open reading frame (ORF) encoding a polypeptide of ˜3000 amino acids comprising both the structural and nonstructural viral proteins.

Upon entry into the cytoplasm of the cell, this RNA is directly translated into a polypeptide of ˜3000 amino acids comprising both the structural and nonstructural viral proteins. This large polypeptide is subsequently processed into 10 individual structural and nonstructural proteins by a combination of host and virally-encoded proteinases (Rice, C. M. (1996) in B. N. Fields, D. M. Knipe and P. M. Howley (eds) Virology 2^{nd} Edition, p 931-960; Raven Press, N.Y.). There are three structural proteins, C, E1 and E2. The P7 protein is of unknown function and is comprised of a highly variable sequence. There are six non-structural proteins. NS2 is a zinc-dependent metalloproteinase that functions in conjunction with a portion of the NS3 protein. NS3 incorporates two catalytic functions (separate from its association with NS2): a serine protease at the N-terminal end, which requires NS4A as a cofactor, and an ATP-ase-dependent helicase function at the carboxyl terminus. NS4A is a tightly associated but non-covalent cofactor of the serine protease.

Following the termination codon at the end of the long ORF, there is a 3′ NTR which roughly consists of three regions: an ˜40 base region which is poorly conserved among various genotypes, a variable length poly(U)/polypyrimidine tract, and a highly conserved 98 base element also called the “3′ X-tail” (Kolykhalov, A. et al (1996) J. Virology 70:3363-3371; Tanaka, T. et al (1995) Biochem Biophys. Res. Commun. 215, 744-749; Tanaka, T. et al (1996) J. Virology 70:3307-3312; Yamada, N. et al (1996) Virology 223:255-261). The 3′ NTR is predicted to form a stable secondary structure which is essential for HCV growth in chimps and is believed to function in the initiation and regulation of viral RNA replication.

The NS5B protein (591 amino acids, 65 kDa) of HCV (Behrens, S. E. et al (1996) EMBO J. 151 2-22), encodes an RNA-dependent RNA polymerase (RdRp) activity and contains canonical motifs present in other RNA viral polymerases. The NS5B protein is fairly well conserved both intra-typically (˜95-98% amino acid (aa) identity across 1b isolates) and inter-typically (˜85% aa identity between genotype 1 a and 1 b isolates). The essentiality of the HCV NS5B RdRp activity for the generation of infectious progeny virions has been formally proven in chimpanzees (A. A. Kolykhalov et al. (2000) Journal of Virology, 74(4): 2046-2051). Thus, inhibition of NS5B RdRp activity (inhibition of RNA replication) is predicted to be useful to treat HCV infection.

Based on the foregoing, there exists a significant need to identify compounds with the ability to inhibit HCV. A general strategy for the development of antiviral agents is to inactivate virally encoded enzymes, including NS5B, that are essential for the replication of the virus.

### SUMMARY OF THE INVENTION

The present invention relates to novel antiviral compounds represented herein below, pharmaceutical compositions comprising such compounds, and methods for the of treatment or prophylaxis of viral (particularly HCV) infection in a subject in need of such therapy with said compounds.

In its principle embodiment, the present invention provides a compound of formula (I):

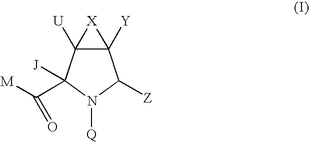

or a pharmaceutically acceptable salt, ester, stereoisomer, tautomer, solvate, prodrug, or combination thereof, wherein:
M at each occurrence is selected from the group consisting of:
a) —OR₁;
b) —NR₁R₂;
c) —SR₁; and
d) —R₁;
   wherein R₁ and R₂ at each occurrence are each independently selected from the group consisting of:
1. hydrogen;
2. deuterium; and
3. —R₃;
   wherein R₃ at each occurrence is selected from the group consisting of:
1) —C₁-C₈ alkyl, —C₂-C₈ alkenyl, —C₂-C₈ alkynyl or —C₃-C₈ cycloalkyl each containing 0, 1, 2, or 3 heteroatoms selected from O, S and N;
2) substituted —C₁-C₈ alkyl, substituted —C₂-C₈ alkenyl, substituted —C₂-C₈ alkynyl or substituted —C₃-C₈ cycloalkyl each containing 0, 1, 2, or 3 heteroatoms selected from O, S and N;
3) heterocyclic;
4) substituted heterocyclic;
5) aryl;
6) substituted aryl;
7) heteroaryl; and
8) substituted heteroaryl;
   or R₁ and R₂ taken together with the nitrogen atom to which they are attached form a substituted or unsubstituted heterocyclic group;
   Q at each occurrence is selected from the group consisting of:
a) —R₁;
b) —C(O)R₁;
c) —C(O)OR₁;
d) —C(O)NR₁R₂;
e) —S(O)ₙR₁, wherein n=0, 1, or 2;
f) —S(O)ₘNR₁R₂, m=1 or 2;
g) —(C═NR₄)NR₁R₂, wherein R₄ is independently R₁;
h) —P(O)R₁R₂;
i) —P(O)(OR₁)(OR₂);
j) —P(O)(NR₁R₂)(NR₂R₄); and
k) —P(O)(NR₁R₂)(OR₄);
   U and Y at each occurrence are each independently selected from the group consisting of:
a) halogen;
b) -M;
c) -Q;
d) —NO₂;
e) —CN;
f) —N₃;
g) —C(R₄)═N—O—R₁;
h) —C(R₄)═N—NR₁R₂;
i) —O-Q; and
j) —N(R₁)-Q;
   Z at each occurrence is independently —R₁;
   X is selected from the group consisting of: —C(A₁A₂)-, —C(A₁A₂)C(B₁B₂)-, or —W—C(A₁A₂)C(B₁B₂)-, wherein A₁, A₂, B₁, B₂ at each occurrence are each independently -M, halogen, and -Q; and W at each occurrence is O, S, or NR₁;
   J at each occurrence is selected from the group consisting of:
a) —C(R₄)═N—O—R₁;
b) —C(R₄)═N—NR₁R₂; and
c) —R₁.

In another embodiment, the present invention provides a pharmaceutical composition comprising a therapeutically effective amount of a compound or combination of compounds of the present invention, or a pharmaceutically acceptable salt form, prodrug, salt of a prodrug, stereoisomer, tautomer, solvate, or combination thereof, in combination with a pharmaceutically acceptable carrier or excipient.

In yet another embodiment, the present invention provides a method of inhibiting the replication of an RNA-containing virus comprising contacting said virus with a therapeutically effective amount of a compound or a combination of compounds of the present invention, or a pharmaceutically acceptable salt, prodrug, salt of a pro drug, stereoisomer, tautomer, solvate, or combination thereof. Particularly, this invention is directed to methods of inhibiting the replication of hepatitis C virus.

In still another embodiment, the present invention provides a method of treating or preventing infection caused by an RNA-containing virus comprising administering to a patient in need of such treatment a therapeutically effective amount of a compound or combination of compounds of the present invention, or a pharmaceutically acceptable salt form, prodrug, salt of a prodrug, stereoisomer, or tautomer, solvate, or combination thereof. Particularly, this invention is directed to methods of treating or preventing infection caused by hepatitis C virus.

Yet another embodiment of the present invention provides the use of a compound or combination of compounds of the present invention, or a therapeutically acceptable salt form, prodrug, salt of a prodrug, stereoisomer or tautomer, solvate, or combination thereof, as defined hereinafter, in the preparation of a medicament for the treatment or prevention of infection caused by RNA-containing virus, specifically hepatitis C virus (HCV).

### DETAILED DESCRIPTION OF THE INVENTION

In a first embodiment of the present invention is a compound of formula (I) as illustrated above, or a pharmaceutically acceptable salt, ester or prodrug thereof.

In a second embodiment of the present invention is the relative stereochemistry of a racemic compound of Formula (I), is represented by formulae (Ia)˜(Id):

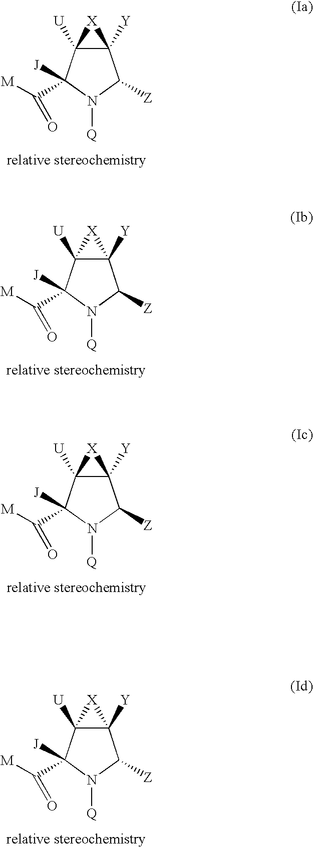

wherein M, Q, Z, X, Y, U, and J are as previously defined.

In a third embodiment of the present invention is the absolute stereochemistry of a chiral compound of Formula (I), is represented by formulae (Iaa)˜(Idd):

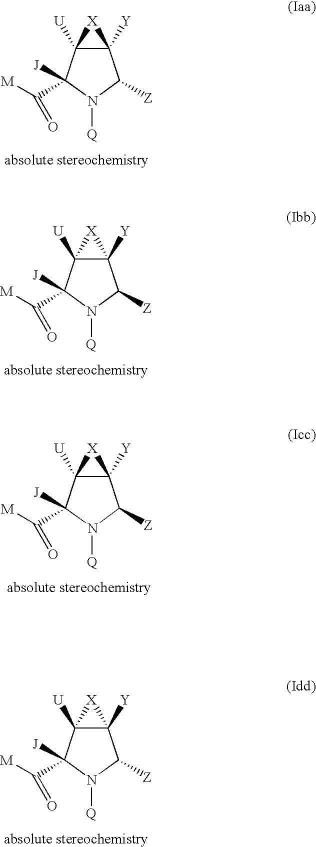

wherein M, Q, Z, X, Y, U, and J are as previously defined.

In a fourth embodiment of the present invention relates to compound of Formula (IIa), or a pharmaceutically acceptable salt, ester or prodrug thereof:

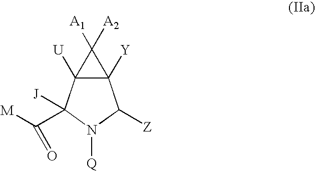

wherein M, Q, Z, Y, U, A₁, A₂, and J are as previously defined.

In a fifth embodiment of the present invention relates to compound of Formula (IIb), or a pharmaceutically acceptable salt, ester or prodrug thereof:

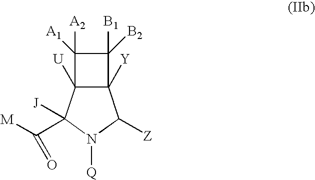

wherein M, Q, Z, Y, U, A₁, A₂, B₁, B₂, and J are as previously defined.

In a sixth embodiment of the present invention relates to compound of Formula (IIc), (IId), or a pharmaceutically acceptable salt, ester or prodrug thereof:

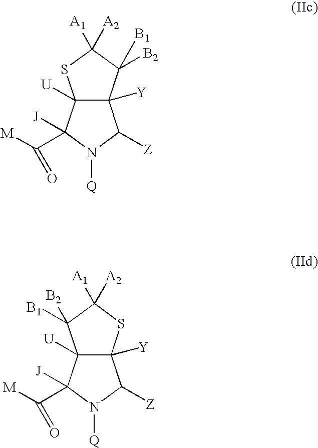

wherein M, Q, Z, Y, U, A₁, A₂, B₁, B₂, and J are as previously defined.

In a seventh embodiment of the present invention relates to compound of Formula (IIIa), (IIIb), or a pharmaceutically acceptable salt, ester or prodrug thereof:

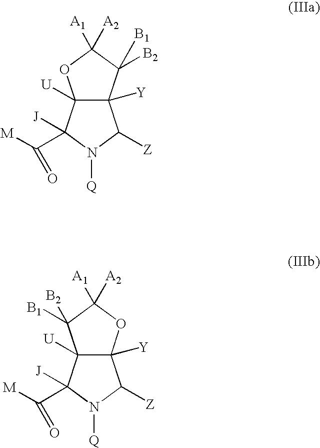

wherein M, Q, Z, U, A₁, A₂, B₁, B₂, Y and J are as previously defined.

In an eighth embodiment of the present invention relates to compound of Formula (IIIc), (IIId), or a pharmaceutically acceptable salt, ester or prodrug thereof:

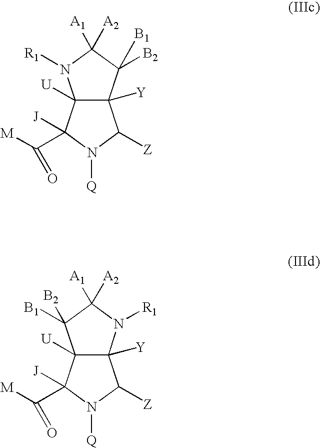

wherein M, Q, Z, U, A₁, A₂, B₁, B₂, R₁, Y and J are as previously defined.

Representative compounds of the present invention are those selected from:

1. Compound of Formula (Ia), wherein M=tert-butoxy, Q=4-tert-butyl-3-methoxybenzoyl, Z=1,3-thiazol-2-yl, X=—CH₂, Y=—CO₂Et, U=hydrogen, J=methyl.
2. Compound of Formula (Ia), wherein M=tert-butoxy, Q=4-tert-butyl-3-methoxybenzoyl, Z=1,3-thiazol-2-yl, X=—CH₂, Y=—CH₂OH, U=hydrogen, J=methyl.
3. Compound of Formula (Ia), wherein M=tert-butoxy, Q=4-tert-butyl-3-methoxybenzoyl, Z=1,3-thiazol-2-yl, X=—CH₂, Y=—CH₂OCH₃, U=hydrogen, J=methyl.
4. Compound of Formula (Ia), wherein M=hydroxyl, Q=4-tert-butyl-3-methoxybenzoyl, Z=1,3-thiazol-2-yl, X=—CH₂, Y=—CH₂OCH₃, U=hydrogen, J=methyl.
5. Compound of Formula (Ia), wherein M=hydroxyl, Q=4-tert-butyl-3-methoxybenzoyl, Z=1,3-thiazol-2-yl, X=—CH₂, Y=—CH₂OCOCF₃, U=hydrogen, J=methyl.
6. Compound of Formula (Ia), wherein M=hydroxyl, Q=4-tert-butyl-3-methoxybenzoyl, Z=1,3-thiazol-2-yl, X=—CH₂, Y=—CH₂OH, U=hydrogen, J=methyl.
7. Compound of Formula (Ia), wherein M=hydroxy, Q=4-tert-butyl-3-methoxybenzoyl, Z=1,3-thiazol-2-yl, X=—CH₂, Y=—CH₂OCH₃, U=hydrogen, J=1H-pyrazol-1-ylmethyl.
8. Compound of Formula (Ia), wherein M=hydroxy, Q=4-tert-butyl-3-methoxybenzoyl, Z=1,3-thiazol-2-yl, X=—CH₂, Y=—C(O)NH₂, U=hydrogen, J=1H-pyrazol-1-ylmethyl.
9. Compound of Formula (Ia), wherein M=hydroxy, Q=4-tert-butyl-3-methoxybenzoyl, Z=1,3-thiazol-2-yl, X=—CH₂, Y=—C(O)NHBn, U=hydrogen, J=1H-pyrazol-1-ylmethyl.
10. Compound of Formula (Ia), wherein M=hydroxy, Q=4-tert-butyl-3-methoxybenzoyl, Z=1,3-thiazol-2-yl, X=—CH₂, Y=—C(O)NHPh, U=hydrogen, J=1H-pyrazol-1-ylmethyl.
11. Compound of Formula (Ia), wherein M=hydroxy, Q=4-tert-butyl-3-methoxybenzoyl, Z=1,3-thiazol-2-yl, X=—CH₂, Y=—CH₂OBn, U=hydrogen, J=1H-pyrazol-1-ylmethyl.
12. Compound of Formula (Ia), wherein M=hydroxy, Q=4-tert-butyl-3-methoxybenzoyl, Z=1,3-thiazol-2-yl, X=—CH₂, Y=—SO₂Ph, U=hydrogen, J=1H-pyrazol-1-ylmethyl.
13. Compound of Formula (Ia), wherein M=hydroxy, Q=4-tert-butyl-3-methoxybenzoyl, Z=1,3-thiazol-2-yl, X=—CH₂, Y=—NHMs, U=hydrogen, J=1H-pyrazol-1-ylmethyl.
14. Compound of Formula (Ia), wherein M=hydroxy, Q=4-tert-butyl-3-methoxybenzoyl, Z=1,3-thiazol-2-yl, X=—CH₂, Y=—CO₂Et, U=hydrogen, J=1H-pyrazol-1-ylmethyl.
15. Compound of Formula (Ia), wherein M=hydroxy, Q=4-tert-butyl-3-methoxybenzoyl, Z=1,3-thiazol-2-yl, X=—CH₂, Y=—CH₂OC(O)NH₂, U=hydrogen, J=1H-pyrazol-1-ylmethyl.
16. Compound of Formula (Ia), wherein M=hydroxy, Q=4-tert-butyl-benzenesulfonyl, Z=1,3-thiazol-2-yl, X=—CH₂, Y=—CH₂OCH₃, U=hydrogen, J=1H-pyrazol-1-ylmethyl.
17. Compound of Formula (Ia), wherein M=hydroxy, Q=4-tert-butyl-phenylcarbamoyl, Z=1,3-thiazol-2-yl, X=—CH₂, Y=—CH₂OCH₃, U=hydrogen, J=1H-pyrazol-1-ylmethyl.
18. Compound of Formula (Ia), wherein M=hydroxy, Q=4-tert-butyl-3-bromobenzoyl, Z=1,3-thiazol-2-yl, X=—CH₂, Y=—CH₂OCH₃, U=hydrogen, J=1H-pyrazol-1-ylmethyl.
19. Compound of Formula (Ia), wherein M=hydroxy, Q=4-tert-butyl-3-vinylbenzoyl, Z=1,3-thiazol-2-yl, X=—CH₂, Y=—CH₂OCH₃, U=hydrogen, J=1H-pyrazol-1-ylmethyl.
20. Compound of Formula (Ia), wherein M=hydroxy, Q=2-fluoro-4-tert-butyl-5-vinylbenzoyl, Z=1,3-thiazol-2-yl, X=—CH₂, Y=—CH₂OCH₃, U=hydrogen, J=1H-pyrazol-1-ylmethyl.
21. Compound of Formula (Ia), wherein M=hydroxy, Q=4-tert-butyl-3-methoxybenzoyl, Z=1,3,4-thiadiazol-2-yl, X=—CH₂, Y=—CH₂OCH₃, U=hydrogen, J=1H-pyrazol-1-ylmethyl.
22. Compound of Formula (Ia), wherein M=hydroxy, Q=4-tert-butyl-3-methoxybenzoyl, Z=5-methylisoxazol-3-yl, X=—CH₂, Y=—CH₂OCH₃, U=hydrogen, J=1H-pyrazol-1-ylmethyl.
23. Compound of Formula (Ia), wherein M=hydroxy, Q=4-tert-butyl-3-methoxybenzoyl, Z=thiophen-2-yl, X=—CH₂, Y=—CH₂OCH₃, U=hydrogen, J=1H-pyrazol-1-ylmethyl.
24. Compound of Formula (Ia), wherein M=hydroxy, Q=4-tert-butyl-3-methoxybenzoyl, Z=thiophen-3-yl, X=—CH₂, Y=—CH₂OCH₃, U=hydrogen, J=1H-pyrazol-1-ylmethyl.
25. Compound of Formula (Ia), wherein M=hydroxy, Q=4-tert-butyl-3-methoxybenzoyl, Z=furan-2-yl, X=—CH₂, Y=—CH₂OCH₃, U=hydrogen, J=1H-pyrazol-1-ylmethyl.
26. Compound of Formula (Ia), wherein M=hydroxy, Q=4-tert-butyl-3-methoxybenzoyl, Z=furan-3-yl, X=—CH₂, Y=—CH₂OCH₃, U=hydrogen, J=1H-pyrazol-1-ylmethyl.
27. Compound of Formula (Ia), wherein M=hydroxy, Q=4-tert-butyl-3-methoxybenzoyl, Z=1,3-oxazol-2-yl, X=—CH₂, Y=—CH₂OCH₃, U=hydrogen, J=1H-pyrazol-1-ylmethyl.
28. Compound of Formula (Ia), wherein M=hydroxy, Q=4-tert-butyl-3-methoxybenzoyl, Z=phenyl, X=—CH₂, Y=—CH₂OCH₃, U=hydrogen, J=1H-pyrazol-1-ylmethyl.
29. Compound of Formula (Ia), wherein M=hydroxy, Q=4-tert-butyl-3-methoxybenzoyl, Z=pyridin-2-yl, X=—CH₂, Y=—CH₂OCH₃, U=hydrogen, J=1H-pyrazol-1-ylmethyl.
30. Compound of Formula (Ia), wherein M=hydroxy, Q=4-tert-butyl-3-methoxybenzoyl, Z=pyridin-3-yl, X=—CH₂, Y=—CH₂OCH₃, U=hydrogen, J=1H-pyrazol-1-ylmethyl.
31. Compound of Formula (Ia), wherein M=hydroxy, Q=4-tert-butyl-3-methoxybenzoyl, Z=pyridin-4-yl, X=—CH₂, Y=—CH₂OCH₃, U=hydrogen, J=1H-pyrazol-1-ylmethyl.
32. Compound of Formula (Ia), wherein M=hydroxy, Q=4-tert-butyl-3-methoxybenzoyl, Z=1,3-thiazol-2-yl, X=—CH₂, Y=—CH₂OCH₃, U=hydrogen, J=—CH═NOMe.
33. Compound of Formula (Ia), wherein M=hydroxy, Q=4-tert-butyl-3-methoxybenzoyl, Z=1,3-thiazol-2-yl, X=—CH₂, Y=—CH₂OCH₃, U=hydrogen, J=—CH═NNMe₂.
34. Compound of Formula (Ia), wherein M=hydroxy, Q=4-tert-butyl-3-methoxybenzoyl, Z=1,3-thiazol-2-yl, X=—CH₂, Y=—CH₂OCH₃, U=hydrogen, J=1,3-thiazol-4-ylmethyl.
35. Compound of Formula (Ia), wherein M=hydroxy, Q=4-tert-butyl-3-methoxybenzoyl, Z=1,3-thiazol-2-yl, X=—CH₂, Y=—CH₂OCH₃, U=hydrogen, J=1,3-thiazol-2-ylmethyl.
36. Compound of Formula (Ia), wherein M=hydroxy, Q=4-tert-butyl-3-methoxybenzoyl, Z=1,3-thiazol-2-yl, X=—CH₂, Y=—CH₂OCH₃, U=hydrogen, J=isoxazol-3-ylmethyl.
37. Compound of Formula (Ia), wherein M=hydroxy, Q=4-tert-butyl-3-methoxybenzoyl, Z=1,3-thiazol-2-yl, X=—CH₂, Y=—CH₂OCH₃, U=hydrogen, J=pyridin-2-ylmethyl.
38. Compound of Formula (Ia), wherein M=hydroxy, Q=4-tert-butyl-3-methoxybenzoyl, Z=1,3-thiazol-2-yl, X=—CH₂, Y=—CH₂OCH₃, U=hydrogen, J=—CH₂NMe₂.
39. Compound of Formula (Ia), wherein M=hydroxy, Q=4-tert-butyl-3-methoxybenzoyl, Z=1,3-thiazol-2-yl, X=—CH₂, Y=—CH₂OCH₃, U=hydrogen, J=—CH₂CH₂NMe₂.
40. Compound of Formula (Ia), wherein M=hydroxy, Q=4-tert-butyl-3-methoxybenzoyl, Z=1,3-thiazol-2-yl, X=—CH₂, Y=—CH₂OCH₃, U=hydrogen, J=—CH₂NHMs.
41. Compound of Formula (Ia), wherein M=hydroxy, Q=4-tert-butyl-3-methoxybenzoyl, Z=1,3-thiazol-2-yl, X=—CH₂, Y=—CH₂OCH₃, U=hydrogen, J=—CH₂CH₂NHMs.
42. Compound of Formula (Ia), wherein M=hydroxy, Q=4-tert-butyl-3-methoxybenzoyl, Z=1,3-thiazol-2-yl-methyl, X=—CH₂, Y=—CH₂OCH₃, U=hydrogen, J=1H-pyrazol-1-ylmethyl.
43. Compound of Formula (Ia), wherein M=hydroxy, Q=4-tert-butyl-3-methoxybenzoyl, Z=isopropyl, X=—CH₂, Y=—CH₂OCH₃, U=hydrogen, J=1H-pyrazol-1-ylmethyl.
44. Compound of Formula (Ia), wherein M=hydroxy, Q=4-tert-butyl-3-methoxybenzoyl, Z=1,3-thiazol-2-yl, X=CF₂, Y=—CH₂OCH₃, U=hydrogen, J=1H-pyrazol-1-ylmethyl.
45. Compound of Formula (Ia), wherein M=hydroxy, Q=4-tert-butyl-3-methoxybenzoyl, Z=1,3-thiazol-2-yl, X=—CH₂CF₂—, Y=—CH₂OCH₃, U=hydrogen, J=1H-pyrazol-1-ylmethyl.
46. Compound of Formula (Ia), wherein M=hydroxy, Q=4-tert-butyl-3-methoxybenzoyl, Z=1,3-thiazol-2-yl, X=—OCH₂CF₂—, Y=—CH₂OCH₃, U=hydrogen, J=1H-pyrazol-1-ylmethyl.

A further embodiment of the present invention includes pharmaceutical compositions comprising any single compound delineated herein, or a pharmaceutically acceptable salt, ester, solvate, or prodrug thereof, with a pharmaceutically acceptable carrier or excipient.

Yet another embodiment of the present invention is a pharmaceutical composition comprising a combination of two or more compounds delineated herein, or a pharmaceutically acceptable salt, ester, solvate, or prodrug thereof, with a pharmaceutically acceptable carrier or excipient.

Yet a further embodiment of the present invention is a pharmaceutical composition comprising any single compound delineated herein in combination with one or more HCV compounds known in the art, or a pharmaceutically acceptable salt, ester, solvate, or prodrug thereof, with a pharmaceutically acceptable carrier or excipient.

It will be appreciated that reference herein to therapy and/or treatment includes, but is not limited to prevention, retardation, prophylaxis, therapy and cure of the disease. It will further be appreciated that references herein to treatment or prophylaxis of HCV infection includes treatment or prophylaxis of HCV-associated disease such as liver fibrosis, cirrhosis and hepatocellular carcinoma.

It will be further appreciated that the compounds of the present invention may contain one or more asymmetric carbon atoms and may exist in racemic, diastereoisomeric, and optically active forms. It will still be appreciated that certain compounds of the present invention may exist in different tautomeric forms. All tautomers are contemplated to be within the scope of the present invention.

It will be further appreciated that the compounds of the invention, or their pharmaceutically acceptable salts, stereoisomers, tautomers, prodrugs or salt of a prodrug thereof, inhibit HCV polymerase, an RNA dependent RNA polymerase, an enzyme essential for HCV viral replication. Compounds of the present invention can be administered as the sole active pharmaceutical agent, or used in combination with one or more agents to treat or prevent hepatitis C infections or the symptoms associated with HCV infection. Other agents to be administered in combination with a compound or combination of compounds of the invention include therapies for disease caused by HCV infection that suppresses HCV viral replication by direct or indirect mechanisms. These include agents such as host immune modulators (for example, interferon-alpha, pegylated interferon-alpha, interferon-beta, interferon-gamma, CpG oligonucleotides and the like), or antiviral compounds that inhibit host cellular functions such as inosine monophosphate dehydrogenase (for example, ribavirin and the like). Also included are cytokines that modulate immune function. Also included are vaccines comprising HCV antigens or antigen adjuvant combinations directed against HCV. Also included are agents that interact with host cellular components to block viral protein synthesis by inhibiting the internal ribosome entry site (IRES) initiated translation step of HCV viral replication or to block viral particle maturation and release with agents targeted toward the viroporin family of membrane proteins such as, for example, HCV P7 and the like. Other agents to be administered in combination with a compound of the present invention include any agent or combination of agents that inhibit the replication of HCV by targeting proteins of the viral genome involved in the viral replication. These agents include but are not limited to other inhibitors of HCV RNA dependent RNA polymerase such as, for example, nucleoside type polymerase inhibitors described in WO01/90121(A2), or U.S. Pat. No. 6,348,587B1 or WO01/60315 or WO01/32153 or non-nucleoside inhibitors such as, for example, benzimidazole polymerase inhibitors described in EP1 162196A1 or WO02/04425.

Accordingly, one aspect of the invention is directed to a method for treating or preventing an infection caused by an RNA-containing virus comprising co-administering to a patient in need of such treatment one or more agents selected from the group consisting of a host immune modulator and a second antiviral agent, or a combination thereof, with a therapeutically effective amount of a compound or combination of compounds of the invention, or a pharmaceutically acceptable salt, stereoisomer, tautomer, prodrug, salt of a prodrug, or combination thereof. Examples of the host immune modulator are, but not limited to, interferon-alpha, pegylated-interferon-alpha, interferon-beta, interferon-gamma, a cytokine, a vaccine, and a vaccine comprising an antigen and an adjuvant, and said second antiviral agent inhibits replication of HCV either by inhibiting host cellular functions associated with viral replication or by targeting proteins of the viral genome.

Further aspect of the invention is directed to a method of treating or preventing infection caused by an RNA-containing virus comprising co-administering to a patient in need of such treatment an agent or combination of agents that treat or alleviate symptoms of HCV infection including cirrhosis and inflammation of the liver, with a therapeutically effective amount of a compound or combination of compounds of the invention, or a pharmaceutically acceptable salt, stereoisomer, tautomer, prodrug, salt of a prodrug, or combination thereof. Yet another aspect of the invention provides a method of treating or preventing infection caused by an RNA-containing virus comprising co-administering to a patient in need of such treatment one or more agents that treat patients for disease caused by hepatitis B (HBV) infection, with a therapeutically effective amount of a compound or a combination of compounds of the invention, or a pharmaceutically acceptable salt, stereoisomer, tautomer, prodrug, salt of a prodrug, or combination thereof. An agent that treats patients for disease caused by hepatitis B (HBV) infection may be for example, but not limited thereto, L-deoxythymidine, adefovir, lamivudine or tenfovir, or any combination thereof. Example of the RNA-containing virus includes, but not limited to, hepatitis C virus (HCV).

Another aspect of the invention provides a method of treating or preventing infection caused by an RNA-containing virus comprising co-administering to a patient in need of such treatment one or more agents that treat patients for disease caused by human immunodeficiency virus (HIV) infection, with a therapeutically effective amount of a compound or a combination of compounds of the invention, or a pharmaceutically acceptable salt, stereoisomer, tautomer, prodrug, salt of a prodrug, or combination thereof. The agent that treats patients for disease caused by human immunodeficiency virus (HIV) infection may include, but is not limited thereto, ritonavir, lopinavir, indinavir, nelfmavir, saquinavir, amprenavir, atazanavir, tipranavir, TMC-114, fosamprenavir, zidovudine, lamivudine, didanosine, stavudine, tenofovir, zalcitabine, abacavir, efavirenz, nevirapine, delavirdine, TMC-125, L-870812, S-1360, enfuvirtide (T-20) or T-1249, or any combination thereof. Example of the RNA-containing virus includes, but not limited to, hepatitis C virus (HCV). In addition, the present invention provides the use of a compound or a combination of compounds of the invention, or a therapeutically acceptable salt form, stereoisomer, or tautomer, prodrug, salt of a prodrug, or combination thereof, and one or more agents selected from the group consisting of a host immune modulator and a second antiviral agent, or a combination thereof, to prepare a medicament for the treatment of an infection caused by an RNA-containing virus in a patient, particularly hepatitis C virus. Examples of the host immune modulator are, but not limited to, interferon-alpha, pegylated-interferon-alpha, interferon-beta, interferon-gamma, a cytokine, a vaccine, and a vaccine comprising an antigen and an adjuvant, and said second antiviral agent inhibits replication of HCV either by inhibiting host cellular functions associated with viral replication or by targeting proteins of the viral genome.

When used in the above or other treatments, combination of compound or compounds of the invention, together with one or more agents as defined herein above, can be employed in pure form or, where such forms exist, in pharmaceutically acceptable salt form, prodrug, salt of a prodrug, or combination thereof. Alternatively, such combination of therapeutic agents can be administered as a pharmaceutical composition containing a therapeutically effective amount of the compound or combination of compounds of interest, or their pharmaceutically acceptable salt form, prodrugs, or salts of the prodrug, in combination with one or more agents as defined hereinabove, and a pharmaceutically acceptable carriers. Such pharmaceutical compositions can be used for inhibiting the replication of an RNA-containing virus, particularly Hepatitis C virus (HCV), by contacting said virus with said pharmaceutical composition. In addition, such compositions are useful for the treatment or prevention of an infection caused by an RNA-containing virus, particularly Hepatitis C virus (HCV).

Hence, further aspect of the invention is directed to a method of treating or preventing infection caused by an RNA-containing virus, particularly a hepatitis C virus (HCV), comprising administering to a patient in need of such treatment a pharmaceutical composition comprising a compound or combination of compounds of the invention or a pharmaceutically acceptable salt, stereoisomer, or tautomer, prodrug, salt of a prodrug, or combination thereof, one or more agents as defined hereinabove, and a pharmaceutically acceptable carrier.

When administered as a combination, the therapeutic agents can be formulated as separate compositions which are given at the same time or within a predetermined period of time, or the therapeutic agents can be given as a single unit dosage form.

Antiviral agents contemplated for use in such combination therapy include agents (compounds or biologicals) that are effective to inhibit the formation and/or replication of a virus in a mammal, including but not limited to agents that interfere with either host or viral mechanisms necessary for the formation and/or replication of a virus in a mammal. Such agents can be selected from another anti-HCV agent; an HIV inhibitor; an HAV inhibitor; and an HBV inhibitor.

Other anti-HCV agents include those agents that are effective for diminishing or preventing the progression of hepatitis C related symptoms or disease. Such agents include but are not limited to immunomodulatory agents, inhibitors of HCV NS3 protease, other inhibitors of HCV polymerase, inhibitors of another target in the HCV life cycle and other anti-HCV agents, including but not limited to ribavirin, amantadine, levovirin and viramidine.

Immunomodulatory agents include those agents (compounds or biologicals) that are effective to enhance or potentiate the immune system response in a mammal. Immunomodulatory agents include, but are not limited to, inosine monophosphate dehydrogenase inhibitors such as VX-497 (merimepodib, Vertex Pharmaceuticals), class I interferons, class II interferons, consensus interferons, asialo-interferons pegylated interferons and conjugated interferons, including but not limited to interferons conjugated with other proteins including but not limited to human albumin. Class I interferons are a group of interferons that all bind to receptor type I, including both naturally and synthetically produced class I interferons, while class II interferons all bind to receptor type II. Examples of class I interferons include, but are not limited to, [alpha]-, [beta]-, [delta]-, [omega]-, and [tau]-interferons, while examples of class II interferons include, but are not limited to, [gamma]-interferons.

Inhibitors of HCV NS3 protease include agents (compounds or biologicals) that are effective to inhibit the function of HCV NS3 protease in a mammal. Inhibitors of HCV NS3 protease include, but are not limited to, those compounds described in WO 99/07733, WO 99/07734, WO 00/09558, WO 00/09543, WO 00/59929, WO 03/064416, WO 03/064455, WO 03/064456, WO 2004/030670, WO 2004/037855, WO 2004/039833, WO 2004/101602, WO 2004/101605, WO 2004/103996, WO 2005/028501, WO 2005/070955, WO 2006/000085, WO 2006/007700 and WO 2006/007708 (all by Boehringer Ingelheim), WO 02/060926, WO 03/053349, WO03/099274, WO 03/099316, WO 2004/032827, WO 2004/043339, WO 2004/094452, WO 2005/046712, WO 2005/051410, WO 2005/054430 (all by BMS), WO 2004/072243, WO 2004/093798, WO 2004/113365, WO 2005/010029 (all by Enanta), WO 2005/037214 (Intermune) and WO 2005/051980 (Schering), and the candidates identified as VX-950, ITMN-191 and SCH 503034.

Inhibitors of HCV polymerase include agents (compounds or biologicals) that are effective to inhibit the function of an HCV polymerase. Such inhibitors include, but are not limited to, non-nucleoside and nucleoside inhibitors of HCV NS5B polymerase. Examples of inhibitors of HCV polymerase include but are not limited to those compounds described in: WO 02/04425, WO 03/007945, WO 03/010140, WO 03/010141, WO 2004/064925, WO 2004/065367, WO 2005/080388 and WO 2006/007693 (all by Boehringer Ingelheim), WO 2005/049622 (Japan Tobacco), WO 2005/014543 (Japan Tobacco), WO 2005/012288 (Genelabs), WO 2004/087714 (IRBM), WO 03/101993 (Neogenesis), WO 03/026587 (BMS), WO 03/000254 (Japan Tobacco), and WO 01/47883 (Japan Tobacco), and the clinical candidates XTL-2125, HCV 796, R-1626 and NM 283.

Inhibitors of another target in the HCV life cycle include agents (compounds or biologicals) that are effective to inhibit the formation and/or replication of HCV other than by inhibiting the function of the HCV NS3 protease. Such agents may interfere with either host or HCV viral mechanisms necessary for the formation and/or replication of HCV. Inhibitors of another target in the HCV life cycle include, but are not limited to, entry inhibitors, agents that inhibit a target selected from a helicase, a NS2/3 protease and an internal ribosome entry site (IRES) and agents that interfere with the function of other viral targets including but not limited to an NS5A protein and an NS4B protein.

It can occur that a patient may be co-infected with hepatitis C virus and one or more other viruses, including but not limited to human immunodeficiency virus (HIV), hepatitis A virus (HAV) and hepatitis B virus (HBV). Thus also contemplated is combination therapy to treat such co-infections by co-administering a compound according to the present invention with at least one of an HIV inhibitor, an HAV inhibitor and an HBV inhibitor.

### DEFINITIONS

Listed below are definitions of various terms used to describe this invention. These definitions apply to the terms as they are used throughout this specification and claims, unless otherwise limited in specific instances, either individually or as part of a larger group.

The term “aryl,” as used herein, refers to a mono- or polycyclic carbocyclic ring system including, but not limited to, phenyl, naphthyl, tetrahydronaphthyl, indanyl, idenyl.

The term “heteroaryl,” as used herein, refers to a mono- or polycyclic aromatic radical having one or more ring atom selected from S, O and N; and the remaining ring atoms are carbon, wherein any N or S contained within the ring may be optionally oxidized. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl.

In accordance with the invention, any of the aryls, substituted aryls, heteroaryls and substituted heteroaryls described herein, can be any aromatic group. Aromatic groups can be substituted or unsubstituted.

The terms “C₁-C₈ alkyl,” or “C₁-C₁₂ alkyl,” as used herein, refer to saturated, straight- or branched-chain hydrocarbon radicals containing between one and eight, or one and twelve carbon atoms, respectively. Examples of C₁-C₈ alkyl radicals include, but are not limited to, methyl, ethyl, propyl, isopropyl, n-butyl, tert-butyl, neopentyl, n-hexyl, heptyl and octyl radicals; and examples of C₁-C₁₂ alkyl radicals include, but are not limited to, ethyl, propyl, isopropyl, n-hexyl, octyl, decyl, dodecyl radicals.

The term “C₂-C₈ alkenyl,” as used herein, refer to straight- or branched-chain hydrocarbon radicals containing from two to eight carbon atoms having at least one carbon-carbon double bond by the removal of a single hydrogen atom. Alkenyl groups include, but are not limited to, for example, ethenyl, propenyl, butenyl, 1-methyl-2-buten-1-yl, heptenyl, octenyl, and the like.

The term “C₂-C₈ alkynyl,” as used herein, refer to straight- or branched-chain hydrocarbon radicals containing from two to eight carbon atoms having at least one carbon-carbon triple bond by the removal of a single hydrogen atom. Representative alkynyl groups include, but are not limited to, for example, ethynyl, 1-propynyl, 1-butynyl, heptynyl, octynyl, and the like.

The term “C₃-C₈-cycloalkyl”, or “C₃-C₁₂-cycloalkyl,” as used herein, refers to a monocyclic or polycyclic saturated carbocyclic ring compound. Examples of C₃-C₈-cycloalkyl include, but not limited to, cyclopropyl, cyclobutyl, cyclopentyl, cyclohexyl, cyclopentyl and cyclooctyl; and examples of C₃-C₁₂-cycloalkyl include, but not limited to, cyclopropyl, cyclobutyl, cyclopentyl, cyclohexyl, bicyclo [2.2.1]heptyl, and bicyclo [2.2.2] octyl.

The term “C₃-C₈ cycloalkenyl”, or “C₃-C₁₂ cycloalkenyl” as used herein, refers to monocyclic or polycyclic carbocyclic ring compound having at least one carbon-carbon double bond. Examples of C₃-C₈ cycloalkenyl include, but not limited to, cyclopropenyl, cyclobutenyl, cyclopentenyl, cyclohexenyl, cycloheptenyl, cyclooctenyl, and the like; and examples of C₃-C₁₂ cycloalkenyl include, but not limited to, cyclopropenyl, cyclobutenyl, cyclopentenyl, cyclohexenyl, cycloheptenyl, cyclooctenyl, and the like.

It is understood that any alkyl, alkenyl, alkynyl and cycloalkyl moiety described herein can also be an aliphatic group, an alicyclic group or a heterocyclic group. An “aliphatic” group is a non-aromatic moiety that may contain any combination of carbon atoms, hydrogen atoms, halogen atoms, oxygen, nitrogen or other atoms, and optionally contain one or more units of unsaturation, e.g., double and/or triple bonds. An aliphatic group may be straight chained, branched or cyclic and preferably contains between about 1 and about 24 carbon atoms, more typically between about 1 and about 12 carbon atoms. In addition to aliphatic hydrocarbon groups, aliphatic groups include, for example, polyalkoxyalkyls, such as polyalkylene glycols, polyamines, and polyimines, for example. Such aliphatic groups may be further substituted.

The term “alicyclic,” as used herein, denotes a monovalent group derived from a monocyclic or bicyclic saturated carbocyclic ring compound by the removal of a single hydrogen atom. Examples include, but not limited to, cyclopropyl, cyclobutyl, cyclopentyl, cyclohexyl, bicyclo [2.2.1]heptyl, and bicyclo [2.2.2] octyl. Such alicyclic groups may be further substituted.

The terms “heterocyclic” or “heterocycloalkyl” can be used interchangeably and referred to a non-aromatic ring or a bi- or tri-cyclic group fused system, where (i) each ring system contains at least one heteroatom independently selected from oxygen, sulfur and nitrogen, (ii) each ring system can be saturated or unsaturated (iii) the nitrogen and sulfur heteroatoms may optionally be oxidized, (iv) the nitrogen heteroatom may optionally be quaternized, (iv) any of the above rings may be fused to an aromatic ring, and (v) the remaining ring atoms are carbon atoms which may be optionally oxo-substituted. Representative heterocyclic groups include, but are not limited to, 1,3-dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, and tetrahydrofuryl. Such heterocyclic groups may be further substituted.

The term “substituted” refers to substitution by independent replacement of one, two, or three or more of the hydrogen atoms thereon with substituents including, but not limited to, —F, —Cl, —Br, —I, —OH, protected hydroxy, —NO₂, —CN, —NH₂, protected amino, oxo, thioxo, —NH—C₁-C₁₂-alkyl, —NH—C₂-C₈-alkenyl, —NH—C₂-C₈-alkynyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₈-alkenyl, —O—C₂-C₈-alkynyl, —O—C₃-C₁₂-cycloalkyl, —O-aryl, —O-heteroaryl, —O-heterocycloalkyl, —C(O)—C₁-C₁₂-alkyl, —C(O)—C₂-C₈-alkenyl, —C(O)—C₂-C₈-alkynyl, —C(O)—C₃-C₁₂-cycloalkyl, —C(O)-aryl, —C(O)-heteroaryl, —C(O)-heterocycloalkyl, —CONH₂, —CONH—C₁-C₁₂-alkyl, —CONH—C₂-C₈-alkenyl, —CONH—C₂-C₈-alkynyl, —CONH—C₃-C₁₂-cycloalkyl, —CONH-aryl, —CONH-heteroaryl, —CONH-heterocycloalkyl, —OCO₂—C₁-C₁₂-alkyl, —OCO₂—C₂-C₈-alkenyl, —OCO₂—C₂-C₈-alkynyl, —OCO₂—C₃-C₁₂-cycloalkyl, —OCO₂-aryl, —OCO₂-heteroaryl, —OCO₂-heterocycloalkyl, —OCONH₂, —OCONH—C₁-C₁₂-alkyl, —OCONH—C₂-C₈-alkenyl, —OCONH—C₂-C₈-alkynyl, —OCONH—C₃-C₁₂-cycloalkyl, —OCONH-aryl, —OCONH-heteroaryl, —OCONH-heterocycloalkyl, —NHC(O)—C₁-C₁₂-alkyl, —NHC(O)—C₂-C₈-alkenyl, —NHC(O)—C₂-C₈-alkynyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₈-alkenyl, —NHCO₂—C₂-C₈-alkynyl, —NHCO₂—C₃-C₁₂-cycloalkyl, —NHCO₂-aryl, —NHCO₂-heteroaryl, —NHCO₂— heterocycloalkyl, —NHC(O)NH₂, —NHC(O)NH—C₁-C₁₂-alkyl, —NHC(O)NH—C₂-C₈-alkenyl, —NHC(O)NH—C₂-C₈-alkynyl, —NHC(O)NH—C₃-C₁₂-cycloalkyl, —NHC(O)NH-aryl, —NHC(O)NH-heteroaryl, —NHC(O)NH-heterocycloalkyl, —NHC(S)NH₂, —NHC(S)NH—C₁-C₁₂-alkyl, —NHC(S)NH—C₂-C₈-alkenyl, —NHC(S)NH—C₂-C₈-alkynyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, —NHC(NH)NH—C₁-C₁₂-alkyl, —NHC(NH)NH—C₂-C₈-alkenyl, —NHC(NH)NH—C₂-C₈-alkynyl, —NHC(NH)NH—C₃-C₁₂-cycloalkyl, —NHC(NH)NH-aryl, —NHC(NH)NH-heteroaryl, —NHC(NH)NH-heterocycloalkyl, —NHC(NH)—C₁-C₁₂-alkyl, —NHC(NH)—C₂-C₈-alkenyl, —NHC(NH)—C₂-C₈-alkynyl, —NHC(NH)—C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₈-alkenyl, —C(NH)NH—C₂-C₈-alkynyl, —C(NH)NH—C₃-C₁₂-cycloalkyl, —C(NH)NH-aryl, —C(NH)NH-heteroaryl, —C(NH)NH— heterocycloalkyl, —S(O)—C₁-C₁₂-alkyl, —S(O)—C₂-C₈-alkenyl, —S(O)—C₂-C₈-alkynyl, —S(O)—C₃-C₁₂-cycloalkyl, —S(O)-aryl, —S(O)-heteroaryl, —S(O)-heterocycloalkyl —SO₂NH₂, —SO₂NH—C₁-C₁₂-alkyl, —SO₂NH—C₂-C₈-alkenyl, —SO₂NH—C₂-C₈-alkynyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₈-alkenyl, —NHSO₂—C₂-C₈-alkynyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, —heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₈-alkenyl, —S—C₂-C₈-alkynyl, —S—C₃-C₁₂-cycloalkyl, —S-aryl, —S-heteroaryl, —S-heterocycloalkyl, or methylthiomethyl. It is understood that the aryls, heteroaryls, alkyls, and the like can be further substituted.

The term “halogen,” as used herein, refers to an atom selected from fluorine, chlorine, bromine and iodine.

The term “hydroxy activating group”, as used herein, refers to a labile chemical moiety which is known in the art to activate a hydroxyl group so that it will depart during synthetic procedures such as in a substitution or an elimination reactions. Examples of hydroxyl activating group include, but not limited to, mesylate, tosylate, triflate, p-nitrobenzoate, phosphonate and the like.

The term “activated hydroxy”, as used herein, refers to a hydroxy group activated with a hydroxyl activating group, as defined above, including mesylate, tosylate, triflate, p-nitrobenzoate, phosphonate groups, for example.

The term “hydroxy protecting group,” as used herein, refers to a labile chemical moiety which is known in the art to protect a hydroxyl group against undesired reactions during synthetic procedures. After said synthetic procedure(s) the hydroxy protecting group as described herein may be selectively removed. Hydroxy protecting groups as known in the art are described generally in T. H. Greene and P. G. M. Wuts, Protective Groups in Organic Synthesis, 3rd edition, John Wiley & Sons, New York (1999). Examples of hydroxyl protecting groups include benzyloxycarbonyl, 4-nitrobenzyloxycarbonyl, 4-bromobenzyloxycarbonyl, 4-methoxybenzyloxycarbonyl, methoxycarbonyl, tert-butoxycarbonyl, isopropoxycarbonyl, diphenylmethoxycarbonyl, 2,2,2-trichloroethoxycarbonyl, 2-(trimethylsilyl)ethoxycarbonyl, 2-furfuryloxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, 1,1-dimethyl-2-propenyl, 3-methyl-3-butenyl, allyl, benzyl, para-methoxybenzyldiphenylmethyl, triphenylmethyl (trityl), tetrahydrofuryl, methoxymethyl, methylthiomethyl, benzyloxymethyl, 2,2,2-trichloroethoxymethyl, 2-(trimethylsilyl)ethoxymethyl, methanesulfonyl, para-toluenesulfonyl, trimethylsilyl, triethylsilyl, triisopropylsilyl, and the like. Preferred hydroxyl protecting groups for the present invention are acetyl (Ac or —C(O)CH₃), benzoyl (Bz or —C(O)C₆H₅), and trimethylsilyl (TMS or —Si(CH₃)₃).

The term “protected hydroxy,” as used herein, refers to a hydroxy group protected with a hydroxy protecting group, as defined above, including benzoyl, acetyl, trimethylsilyl, triethylsilyl, methoxymethyl groups, for example.

The term “hydroxy prodrug group”, as used herein, refers to a promoiety group which is known in the art to change the physicochemical, and hence the biological properties of a parent drug in a transient manner by covering or masking the hydroxy group. After said synthetic procedure(s), the hydroxy prodrug group as described herein must be capable of reverting back to hydroxy group in vivo. Hydroxy prodrug groups as known in the art are described generally in Kenneth B. Sloan, Prodrugs, Topical and Ocular Drug Delivery, (Drugs and the Pharmaceutical Sciences; Volume 53), Marcel Dekker, Inc., New York (1992).

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the art are described generally in T. H. Greene and P. G. M. Wuts, Protective Groups in Organic Synthesis, 3rd edition, John Wiley & Sons, New York (1999). Examples of amino protecting groups include, but are not limited to, t-butoxycarbonyl, 9-fluorenylmethoxycarbonyl, benzyloxycarbonyl, and the like.

The term “leaving group” means a functional group or atom which can be displaced by another functional group or atom in a substitution reaction, such as a nucleophilic substitution reaction. By way of example, representative leaving groups include chloro, bromo and iodo groups; sulfonic ester groups, such as mesylate, tosylate, brosylate, nosylate and the like; and acyloxy groups, such as acetoxy, trifluoroacetoxy and the like.

The term “protected amino,” as used herein, refers to an amino group protected with an amino protecting group as defined above.

The term “aprotic solvent,” as used herein, refers to a solvent that is relatively inert to proton activity, i.e., not acting as a proton-donor. Examples include, but are not limited to, hydrocarbons, such as hexane and toluene, for example, halogenated hydrocarbons, such as, for example, methylene chloride, ethylene chloride, chloroform, and the like, heterocyclic compounds, such as, for example, tetrahydrofuran and N-methylpyrrolidinone, and ethers such as diethyl ether, bis-methoxymethyl ether. Such compounds are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of aprotic solvents may be found in organic chemistry textbooks or in specialized monographs, for example: Organic Solvents Physical Properties and Methods of Purification, 4th ed., edited by John A. Riddick et al, Vol. II, in the Techniques of Chemistry Series, John Wiley & Sons, NY, 1986.

The term “protic solvent” as used herein, refers to a solvent that tends to provide protons, such as an alcohol, for example, methanol, ethanol, propanol, isopropanol, butanol, t-butanol, and the like. Such solvents are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of protogenic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The synthesized compounds can be separated from a reaction mixture and further purified by a method such as column chromatography, high pressure liquid chromatography, or recrystallization. As can be appreciated by the skilled artisan, further methods of synthesizing the compounds of the Formula herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2^{nd} Ed. Wiley-VCH (1999); T. W. Greene and P. G. M. Wuts, Protective Groups in Organic Synthesis, 3rd Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The term “subject” as used herein refers to an animal. Preferably the animal is a mammal. More preferably the mammal is a human. A subject also refers to, for example, dogs, cats, horses, cows, pigs, guinea pigs, fish, birds and the like.

The compounds of this invention may be modified by appending appropriate functionalities to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. Tautomers may be in cyclic or acyclic. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid. Examples of pharmaceutically acceptable salts include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention. “Prodrug”, as used herein means a compound which is convertible in vivo by metabolic means (e.g. by hydrolysis) to a compound of the invention. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991); Bundgaard, et al., Journal of Drug Deliver Reviews, 8:1-38 (1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

The present invention also relates to solvates of the compounds of Formula (I), for example hydrates.

This invention also encompasses pharmaceutical compositions containing, and methods of treating viral infections through administering, pharmaceutically acceptable prodrugs of compounds of the invention. For example, compounds of the invention having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the invention. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

### Pharmaceutical Compositions

The pharmaceutical compositions of the present invention comprise a therapeutically effective amount of a compound of the present invention formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. No. 5,767,068 to VanDevanter et al., U.S. Pat. No. 5,508,269 to Smith et al., and WO 98/43650 by Montgomery, all of which are incorporated herein by reference). A discussion of pulmonary delivery of antibiotics is also found in U.S. Pat. No. 6,014,969, incorporated herein by reference.

According to the methods of treatment of the present invention, viral infections, conditions are treated or prevented in a patient such as a human or another animal by administering to the patient a therapeutically effective amount of a compound of the invention, in such amounts and for such time as is necessary to achieve the desired result.

By a “therapeutically effective amount” of a compound of the invention is meant an amount of the compound which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the compound described above may range from about 0.1 mg/Kg to about 500 mg/Kg, preferably from about 1 to about 50 mg/Kg. Effective doses will also vary depending on route of administration, as well as the possibility of co-usage with other agents. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

The total daily dose of the compounds of this invention administered to a human or other animal in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens according to the present invention comprise administration to a patient in need of such treatment from about 10 mg to about 1000 mg of the compound(s) of this invention per day in single or multiple doses.

The compounds of the Formula described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.1 to about 500 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with pharmaceutically excipients or carriers to produce a single dosage form will vary depending upon the host treated and the particular mode of administration. A typical preparation will contain from about 5% to about 95% active compound (w/w). Alternatively, such preparations may contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

When the compositions of this invention comprise a combination of a compound of the invention described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The said “additional therapeutic or prophylactic agents” includes but not limited to, immune therapies (eg. interferon), therapeutic vaccines, antifibrotic agents, anti-inflammatory agents such as corticosteroids or NSAIDs, bronchodilators such as beta-2 adrenergic agonists and xanthines (e.g. theophylline), mucolytic agents, anti-muscarinics, anti-leukotrienes, inhibitors of cell adhesion (e.g. ICAM antagonists), anti-oxidants (eg N-acetylcysteine), cytokine agonists, cytokine antagonists, lung surfactants and/or antimicrobial and anti-viral agents (eg ribavirin and amantidine). The compositions according to the invention may also be used in combination with gene replacement therapy.

Unless otherwise defined, all technical and scientific terms used herein are accorded the meaning commonly known to one of ordinary skill in the art. All publications, patents, published patent applications, and other references mentioned herein are hereby incorporated by reference in their entirety.

### Abbreviations

Abbreviations which may be used in the descriptions of the scheme and the examples that follow are:
Ac for acetyl;
AcOH for acetic acid;
AIBN for azobisisobutyronitrile;
BINAP for 2,2′-bis(diphenylphosphino)-1,1′-binaphthyl;
Boc₂O for di-tert-butyl-dicarbonate;
Boc for t-butoxycarbonyl;
Bpoc for 1-methyl-1-(4-biphenylyl)ethyl carbonyl;
Bz for benzoyl;
Bn for benzyl;
BocNHOH for tert-butyl N-hydroxycarbamate;
t-BuOK for potassium tert-butoxide;
Bu₃SnH for tributyltin hydride;
BOP for (benzotriazol-1-yloxy)tris(dimethylamino)phosphonium Hexafluorophosphate;
Brine for sodium chloride solution in water;
CDI for carbonyldiimidazole;
CH₂Cl₂ for dichloromethane;
CH₃ for methyl;
CH₃CN for acetonitrile;
Cs₂CO₃ for cesium carbonate;
CuCl for copper (I) chloride;
CuI for copper (I) iodide;
dba for dibenzylidene acetone;
dppb for diphenylphosphino butane;
DBU for 1,8-diazabicyclo[5.4.0]undec-7-ene;
DCC for N,N′-dicyclohexylcarbodiimide;
DEAD for diethylazodicarboxylate;
DIAD for diisopropyl azodicarboxylate;
DIPEA or (i-Pr)₂EtN for N,N,-diisopropylethyl amine;
Dess-Martin periodinane for 1,1,1-tris(acetyloxy)-1,1-dihydro-1,2-benziodoxol-3-(1H)-one;
DMAP for 4-dimethylaminopyridine;
DME for 1,2-dimethoxyethane;
DMF for N,N-dimethylformamide;
DMSO for dimethyl sulfoxide;
DPPA for diphenylphosphoryl azide;
EDC for N-(3-dimethylaminopropyl)-N′-ethylcarbodiimide;
EDC HCl for N-(3-dimethylaminopropyl)-N′-ethylcarbodiimide hydrochloride;
EtOAc for ethyl acetate;
EtOH for ethanol;
Et₂O for diethyl ether; HATU for O-(7-azabenzotriazol-1-yl)-N,N,N′,N′,-tetramethyluronium
Hexafluorophosphate;
HCl for hydrogen chloride;
HOBT for 1-hydroxybenzotriazole;
K₂CO₃ for potassium carbonate;
n-BuLi for n-butyl lithium;
i-BuLi for i-butyl lithium;
t-BuLi for t-butyl lithium;
PhLi for phenyl lithium;
LDA for lithium diisopropylamide;
TMEDA for N,N,N′,N′-tetramethylethylenediamine;
LiTMP for lithium 2,2,6,6-tetramethylpiperidinate;
MeOH for methanol;
Mg for magnesium;
MOM for methoxymethyl;
Ms for mesyl or —SO₂—CH₃;
Ms₂O for methanesulfonic anhydride or mesyl-anhydride;
NaN(TMS)₂ for sodium bis(trimethylsilyl)amide;
NaCl for sodium chloride;
NaH for sodium hydride;
NaHCO₃ for sodium bicarbonate or sodium hydrogen carbonate;
Na₂CO₃ sodium carbonate;
NaOH for sodium hydroxide;
Na₂SO₄ for sodium sulfate;
NaHSO₃ for sodium bisulfite or sodium hydrogen sulfite;
Na₂S₂O₃ for sodium thiosulfate;
NH₂NH₂ for hydrazine;
NH₄HCO₃ for ammonium bicarbonate;
NH₄Cl for ammonium chloride;
NMMO for N-methylmorpholine N-oxide;
NaIO₄ for sodium periodate;
Ni for nickel;
OH for hydroxyl;
OsO₄ for osmium tetroxide;
TEA or Et₃N for triethylamine;
TFA for trifluoroacetic acid;
THF for tetrahydrofuran;
TPP or PPh₃ for triphenylphosphine;
Troc for 2,2,2-trichloroethyl carbonyl;
Ts for tosyl or —SO₂—C₆H₄CH₃;
Ts₂O for tolylsulfonic anhydride or tosyl-anhydride;
TsOH for p-tolylsulfonic acid;
Pd for palladium;
Ph for phenyl;
POPd for dihydrogen dichlorobis(di-tert-butylphosphinito-κP)palladate(II);
Pd₂(dba)₃ for tris(dibenzylideneacetone) dipalladium (0);
Pd(PPh₃)₄ for tetrakis(triphenylphosphine)palladium (0);
PdCl₂(Ph₃P)₂ for trans-dichlorobis(triphenylphosphine)palladium (II);
Pt for platinum;
Rh for rhodium;
Ru for ruthenium;
TBS for tert-butyl dimethylsilyl; or
TMS for trimethylsilyl;
TMSCl for trimethylsilyl chloride.

### Synthetic Methods

The compounds and processes of the present invention will be better understood in connection with the following synthetic schemes that illustrate the methods by which the compounds of the invention may be prepared.

The compounds of the present invention may be prepared via several different synthetic routes. The most straightforward method, as shown in Scheme 1, in which and following schemes M, Q, Z, G, X, Y, U, W and J are as previously defined, includes a ring closure between an imine intermediate (1-2) and a suitable olefin (1-2.1, wherein X¹ and X² at each occurrence are each independently a halogen, carbon or heteroatom-centered group) promoted by a Lewis acid such as but not limited to lithium bromide, titanium (IV) chloride, boron trifluoride etherate, silver acetate, or the like; or by a base such as but not limited to triethylamine, DBU, pyridine, potassium carbonate, sodium bicarbonate, lithium tert-butoxide, or the like; or a combination of a Lewis acid and a suitable base such as but not limited to lithium bromide and triethylamine, in an aprotic solvent at a temperature typically between −20° C. and 100° C. The preferred temperature is 0° C. to room temperature. (1-2.1) is a suitably substituted olefin, with one or more substituents as electron-withdrawing-group or electron-deficient heteroaryl, such as but not limited to methyl 4-bromo-2-butenoate, methyl 4,4-dibromo-2-butenoate, methyl 4-bromo-2-pentenoate, methyl 4-bromo-4-fluoro-2-butenoate, methyl 5-bromo-2-pentenoate, methyl 5-bromo-5,5-difluoro-2-pentenoate, methyl 5-bromo-4,4-difluoro-2-pentenoate, methyl 5-bromo-4,5-difluoro-2-pentenoate, methyl 3-(2-Bromoethoxy) acrylate, ethyl 3-(2-Bromoethoxy) acrylate ester, ethyl 3-(2-Bromo-1,1-difluoro-ethoxy)-acrylate, ethyl 3-(2-Bromo-2,2-difluoro-ethoxy)-acrylate, 4-bromo-but-2-enenitrile, 5-bromo-pent-3-en-2-one, or the like. Imine (1-2) can be obtained by condensation of a α-amino carbonyl species, typically an amino acid derivative such as t-butyl 2-amino-3-(1,3-thiazol-4-yl)-propanoate, t-butyl 3-(1H-pyrazol-1-yl)-propanoate, benzyl 2-amino-3-(t-butyldimethylsilyloxy)-propanoate, t-butyl 2-amino-4-methyl-pentanoate, t-butyl 2-amino-3-(tert-butyl-dimethylsiloxy)-propanoate, benzyl 2-amino-3-(t-butyl-dimethylsiloxy)-propanoate, t-butyl 2-amino-3-benzyloxy-propanoate, t-butyl 2-amino-3-benzyloxycarbonyloxy-propanoate, t-butyl 2-aminopropanoate, t-butyl 2-aminoacetate, or the like, with an aldehyde (1-1.1) such as 2-formyl-1,3-thiazole, 1,3-thiazol-2-yl-acetaldehyde, 2-methylpropionaldehyde, benzaldehyde, or the like, promoted by a water-scavenger such as but not limited to magnesium sulfate, molecular sieves, methyl orthoformate, or the like; optionally in the presence of an acid such as but not limited to acetic acid, p-toluenesulfonic acid, lithium bromide, or the like, or a base such as but not limited to triethylamine, pyridine, sodium bicarbonate, or the like, or a combination of an acid and a suitable base; in an aprotic solvent at a temperature typically between −20° C. and 100° C. The preferred temperature is 0° C. to room temperature. Pyrrolidine (1-3) is converted to intermediate of formula (1-4) by derivatizing the reactive secondary amine with reagent (1-3.1), wherein LG is a leaving group such as but not limited to hydrogen, chloride, bromide, OMs, benzotriazolyl, hydroxyl, acetoxy, t-butoxycarboxy, or the like, in the presence of a base such as but not limited to triethylamine, pyridine, sodium bicarbonate, or the like, optionally in the presence of an condensation reagent which is known in the art such as EDC, HATU, or the like, in an aprotic solvent at a temperature typically between 0° C. and 100° C., preferably at room temperature.

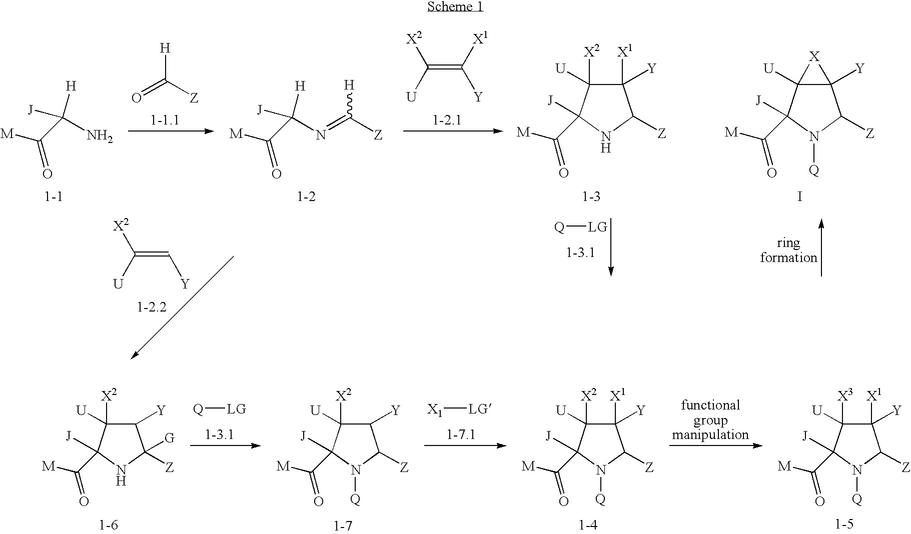

Alternatively as shown in Scheme 1, the intermediate (1-4) may be prepared from intermediate (1-7) by extracting a proton with a strong base such as but not limited to LDA, t-BuLi, PhLi, LiTMP, or the like, optionally in the presence of a lithium chelating agent, which is known in the art, such as TMEDA or the like, in an aprotic solvent or a combination of aprotic solvents at a temperature typically between −78° C. and room temperature, followed by trapping the resulted carbanion with reagent (1-7.1) in an aprotic solvent or a combination of aprotic solvents at a temperature typically between −78° C. and 100° C. The carbanion trapping reagent (1-7.1) is a reactive species, selected from a group such as but not limited to methyl iodide, acetyl chloride, benzyl bromide, allyl bromide, benzoyl chloride, propargyl bromide, 1,4-dichloro-2-butyne, 2-chloromethyl-3-chloro-1-propene, cis-4-chloro-1-(t-butyldimethylsilyloxy)-2-butene, N-fluorobenzenesulfonimide, NCS, 2-formylpyridine, methoxymethyl chloride, or the like. The intermediate (1-7) may be prepared by a two steps procedure: 1) cyclization of an imine (1-2) and an olefin (1-2.2) to give a pyrrolidine intermediate (1-6); and 2) condensation of (1-6) with reagent (1-3.1); using the conditions described above.

As shown in Scheme 1, it may be necessary to convert intermediate (1-4) to (1-5, wherein X³ is a halogen, carbon or heteroatom-centered group, such as but not limited to bromo, bromomethyl, methanesulfonylmethyl, hydroxy, methylamino, acetamino, 3-acetoxy-1-propen-1-yl, or the like) through one-step or steps of functional group manipulation, which are known in the art, including but not limited to oxidation, reduction, protection, deprotection, hydrogenation, alkylation, hydrolysis, activation, Wittig olefination, substitution, elimination, or the like. Intermediate (1-5) can then be converted to the compound of the invention through an intramolecular cyclization of a moiety between C3-position and C4-position of the pyrrolidine ring, which are known in the art, including but not limited to anionic chemistry, cationic chemistry or radical chemistry, some examples are detailed in Scheme 2.

Scheme 2 describes methods that can be used to promote the intramolecular cyclization between C3 to C4 of the pyrrolidine core. The C5-proton of intermediate (2-1, wherein E is a carbon or heteroatom centered moiety; q is an integer from 1 to 3, and LG is as defined previously) is extracted by a base which can be added externally or generated internally from the LG-group, and optionally in the presence of a transitional metal catalyst such as but not limited to Pd(PPh₃)₄, Pd₂(dba)₃, Pd(OAc)₂, or the like; and a ligand such as but not limited to dppb, AsPh₃, tris-(2-furyl)phosphine, trimethyl phosphite, or the like, in an aprotic solvent such as but not limited to THF, DMF, acetonitrile, toluene, or the like, at temperature typically from −20° C. to refluxing depending on the solvent used, for a period of time from 1 hour to 5 days. The externally added base includes but not limited to DBU, LDA, sodium hydride, potassium hydride, DMAP, or the like. The carbanion thus generated at C5 can attack a moiety at C4 in a nucleophilic fashion which is known in the art to form a carbon-carbon or carbon-heteroatom bond in (2-2) with departure of the LG group. Optionally this intramolecular cyclization process can happen with an expansion of forming ring size in the presence of an alkylating reagent (2-1.1, wherein LG₁ and LG₂ are each independently LG, E₁ is independently E and r is independently q) such as but not limited to 1,3-dichloroacetone, 3-chloro-2-chloromethyl-1-propene, 2-bromomethyl-oxirane, carbonic acid 2-t-butoxycarbonyloxymethyl-allyl ester t-butyl ester, carbonic acid 4-t-butoxycarbonyloxy-but-2-enyl ester t-butyl ester, or the like.

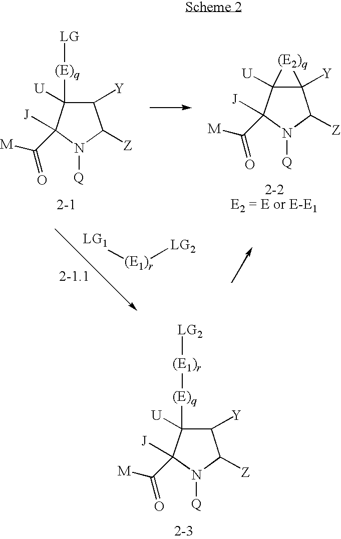

It will be appreciated that compounds and/or intermediates of the invention which exist as diastereoisomers may optionally be separated by techniques well known in the art, for example by column chromatography.

It will be appreciated that racemic compounds and/or intermediates of the invention may be optionally resolved into their individual enantiomers. Such resolutions may conveniently be accomplished by standard methods known in the art. For example, a racemic compound and/or intermediates of the invention may be resolved by chiral preparative HPLC. Alternatively, racemic compounds and/or intermediates of the invention which contain an appropriate acidic or basic group, such as a carboxylic acid group or amine group may be resolved by standard diastereoisomeric salt formation with a chiral base or acid reagent respectively as appropriate. Such techniques are well established in the art. For example, a racemic compound of Formula (1-3) or (1-6) may be resolved by treatment with a chiral acid such as (R)-(−)-1,1′-binaphthyl-2,2′-diyl-hydrogen phosphate, in a suitable solvent, for example dichloromethane, isopropanol or acetonitrile. The enantiomer of Formula (1-3) or (1-6) may then be released by treating the salt with a suitable base, for example triethylamine, in a suitable solvent, for example methyl tert-butyl ether. Individual enantiomers of Formula (1-3) or (1-6) may then be progressed to an enantiomeric compound of the invention by the chemistry described above in respect of racemic compounds.

It will also be appreciated that individual enantiomeric compounds of Formula (I-3) or (1-6) may be prepared by general methods of asymmetric synthesis using, where appropriate, chiral auxiliaries or chiral catalytic reagents and additionally performing any suitable functional group interconversion step as hereinbefore described, including the addition or removal of any such chiral auxiliary. Such general methods of asymmetric synthesis are well known in the art and include, but are not restricted to, those described in “Asymmetric Synthesis,” Academic Press, 1984 and/or “Chiral Auxiliaries and Ligands in Asymmetric Synthesis”, Wiley, 1995. For example, suitable general chiral auxiliaries include chiral alcohols such as menthol or 1-phenylethanol; chiral oxazolidinones such as 4-benzyloxazolidin-2-one or 4-isopropyloxazolidin-2-one; chiral sultams such as camphor sultam; or chiral amines such as 1-phenylethylamine or 2-amino-2-phenylethanol. Suitable general chiral catalytic reagents include chiral basic amines and chiral ligands such as N-methylephedrine, 1-phenyl-2-(1-pyrrolidinyl)-1-propanol, 3-(dimethylamino)-1,7,7-trimethylbicyclo[2.2.1]-heptan-2-ol, 3,4-bis(diphenylphosphanyl)-1-(phenylmethyl)-pyrrolidine, chinchonine, chinchonidine, sparteine, hydroquinine or quinine, BINAP or chiral bis(oxazoline) (BOX) ligands and derivatives, optionally in the presence of a metal salt, for example AₐB_{b} where A is silver, cobalt, zinc, titanium, magnesium, or manganese, and B is halide (for example chloride or bromide), acetate, trifluoroacetate, p-toluenesulfonate, trifluoromethylsulfonate, hexafluorophosphate or nitrate, and ₐ, and _{b}, are 1, 2, 3 or 4, and optionally in the presence of a base, for example triethylamine. All of these chiral auxiliaries or chiral catalytic reagents are well described in the art. General illustrative examples of the preparation of various chiral pyrrolidines by asymmetric synthesis using chiral auxiliaries or chiral catalytic reagents include, but are not limited to, those described in Angew. Chem. Int. Ed., (2002), 41, 4236; Chem. Rev., (1998), 98, 863; J. Am. Chem. Soc., (2002), 124, 13400; J. Am. Chem. Soc., (2003), 125, 10175; Org. Lett., (2003), 5, 5043; Tetrahedron, (1995), 51, 273; Tetrahedron: Asymm., (1995), 6, 2475; Tetrahedron: Asymm., (2001), 12, 1977; Tetrahedron: Asymm., (2002), 13, 2099 and Tet. Lett., (1991), 41, 5817.

In a particular aspect, a chiral pyrrolidine compound of Formula (2-1a) in Scheme 3 in which Y¹ represents —CO₂L or —CO₂L¹ wherein L represents hydrogen or alkyl, L¹ represents a chiral auxiliary, and M, Z, U, X¹, X² and J are as defined above, and * denotes an enantioenriched chiral centre can be prepared by reaction of a compound of Formula (1-2), as hereinbefore defined, with a compound of Formula (1-2.3a) in which Y¹ represents a chiral ester group —CO₂L¹ wherein L¹ represents a chiral auxiliary and thereafter optionally carrying out any conversion of —CO₂L¹ into —CO₂L by standard methods for removal of chiral auxiliaries. Such chiral ester —CO₂L¹ may be derived from a chiral alcohol L¹OH, for example menthol, by standard esterification techniques. Preferably, the reaction of a compound of Formula (1-2) with a compound of Formula (1-2.3a) is carried out in an aprotic solvent, for example THF or acetonitrile, optionally in the presence of a Lewis acid catalyst, such as lithium bromide or silver acetate, and a base, such as triethylamine, DBU or tetramethyl guanidine. Alternatively, the reaction is carried out in an aprotic solvent, for example THF or acetonitrile, in the presence of an acid, such as acetic acid, or the reaction may be carried out by heating compounds of Formula (1-2) and (1-2.3a) in a suitable solvent, for example toluene, xylene or acetonitrile in the absence of a catalyst. The preparation of compounds analogous to those of Formula (1-2.3a) and (2-1a) is described in Tetrahedron: Asymm., (1995), 6, 2475. The construction of enantiopure pyrrolidine ring system via asymmetric [3+2]-cycloaddition of azomethine ylides was recently reviewed (Pandey, G. et al, Chem. Rev. 2006, 106, 4484) and incorporated herein by reference.

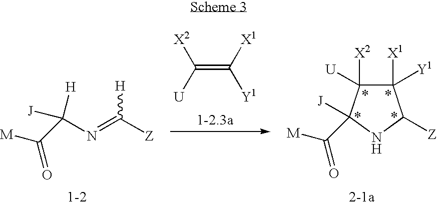

Optional removal of a chiral auxiliary from a group in which Y¹ represents —CO₂L¹ to afford a group in which V represents —CO₂L is readily accomplished by standard methods, for example treatment with a hydrolytic reagent such as sodium hydroxide or an alkoxide such as sodium methoxide as appropriate, in a suitable solvent such as methanol.

In a further aspect, a chiral pyrrolidine compound of Formula (2-1b) in Scheme 4 in which Y¹ represents —CO₂L wherein L represents hydrogen or alkyl, and M, Z, U, X¹, X² and J are as defined above, and * denotes an enantioenriched chiral centre can be prepared by reaction of a compound of Formula (1-2) with a compound of Formula (1-2.3b) as herein before defined, under asymmetric reaction conditions. It will be appreciated by those skilled in the art that such asymmetric reaction conditions may be afforded by, for example, the inclusion in the reaction mixture of a chiral catalytic reagent as herein before defined.

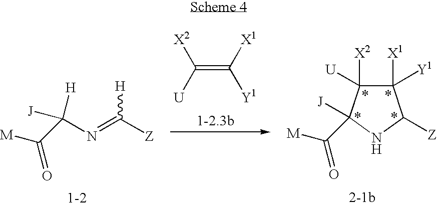

In one aspect, the reaction is carried out in the presence of a suitable chiral catalytic reagent, for example (−)-N-methylephedrine, and a suitable metal salt, for example manganese (II) bromide, in a suitable solvent, for example acetonitrile. Preferably the reaction is carried out at a temperature in the range −30° C. to room temperature, suitably at −20° C.

In an alternative aspect, the reaction is carried out in the presence of a suitable chiral catalytic reagent, for example (S)-BINAP, and a suitable metal salt, for example silver acetate, in the presence of a suitable base, for example diisopropylethylamine, in a suitable solvent, for example acetonitrile optionally co-solvated with toluene. Preferably the reaction is carried out at a temperature in the range −15° C. to room temperature, suitably at −5° C.

Optionally, the major chiral diastereoisomer of a compound of Formula (2-1a) or Formula (2-1b) arising from such an asymmetric reaction may be further enantio-enriched by conventional purification techniques well known in the art, for example by chromatography, or by fractional crystallization. A favourable crystallization method is the fractional crystallization of a salt of the major chiral diastereoisomer, for example the hydrochloride salt or the (R)-(−)-1,1′-binaphthyl-2,2′-diyl-hydrogen phosphate salt. The hydrochloride salt of a compound of Formula (2-1a) or Formula (2-1b) may be prepared by treating a compound of Formula (2-1a) or Formula (2-1b) with anhydrous hydrogen chloride in a suitable solvent, for example diethyl ether. Preferably the reaction is carried out at a temperature in the range −10 to 10° C. Resolution of the racemic mixture can be carried out using (R)-(−)-1,1′-binaphthyl-2,2′-diyl-hydrogen phosphate salt as described above for the resolution of a racemic compound of Formula (1-3) or (1-5).

It will be appreciated that, with appropriate manipulation and protection of any chemical functionality, synthesis of compounds of the invention is accomplished by methods analogous to those above and to those described in the Experimental section. Suitable protecting groups can be found, but are not restricted to, those found in T W Greene and P G M Wuts “Protective Groups in Organic Synthesis”, 3rd Ed (1999), J Wiley and Sons.

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

### EXAMPLES

The compounds and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Although the invention has been described with respect to various preferred embodiments, it is not intended to be limited thereto, but rather those skilled in the art will recognize that variations and modifications may be made therein which are within the spirit of the invention and the scope of the appended claims.

### Example 1

### Compound of Formula (Ia) Wherein M=tert-butoxy, Q=4-tert-butyl-3-methoxybenzoyl, Z=1,3-thiazol-2-yl, X=—CH₂, Y=—CO₂Et, U=hydrogen, J=methyl

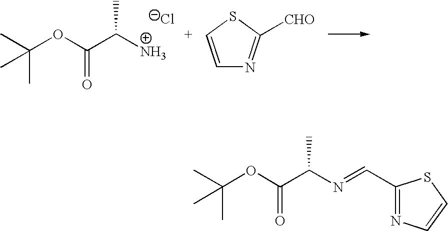

Step 1a. To a 100 mL round-bottomed flask are added commercially available 2-formylthiazole (2.0 g, 17.7 mmol), L-alanine t-butyl ester hydrochloride (3.2 g, 17.7 mmol), 4 Å molecular sieve (5.0 g), anhydrous methylene chloride (50 mL) respectively at 0° C. followed by the addition of triethylamine (2.96 mL, 21.2 mmol) dropwisely. After stirred for 1 h at 0° C. for 1 h, the reaction mixture is warmed up to room temperature and stirred vigorously overnight. The mixture is filtered through a pad of celite and the insoluble is washed thoroughly with methylene chloride. The combined filtrate and washings are concentrated in vacuo, and then treated with diethyl ether (300 mL). The white precipitate is filtered off and the filtrate is concentrated to give a brown oil 4.99 g which is used directly for next step without further purification.

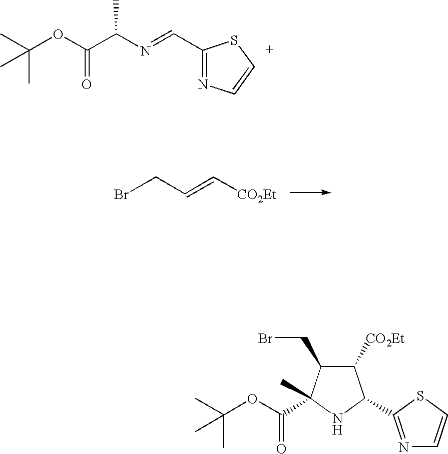

Step 1b. To a 100 mL 3-necked round-bottomed flask are added the compound from step 1a (2.5 g, 8.85 mmol), the commercially available ethyl 4-bromocrotonate (1.37 mL, 10.6 mmol), lithium bromide (1.15 g, 13.3 mmol), and freshly distilled THF (40 mL) respectively at 0° C. followed by the addition of triethylamine (3.7 mL, 26.6 mmol) dropwisely. After stirred for 1 h at 0° C. for 1 h, the reaction mixture is warmed up to room temperature and stirred for another 7 h. After being diluted with methylene chloride, the solution is washed with saturated NaHCO₃ and brine, and dried over anhydrous Na₂SO₄, filtered, and concentrated. The residue is purified by flash chromatography with EtOAc/Hexanes (10-100%) to give the desired product (380 mg, 10% yield).

ESIMS m/z=433.13 [M+H]⁺.

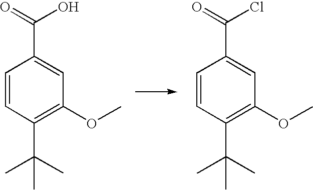

Step 1c. A mixture of the commercially available 4-t-butyl-3-methoxybenzoic acid (2.082 g, 10.0 mmol) in thionyl chloride (5.0 mL) is refluxed for 2.5 hours before being evaporated. Toluene (twice) is added to the residue and the mixture is evaporated. The residue is dried in vacuum to get a crystalline (2.258 g, 99.6%).

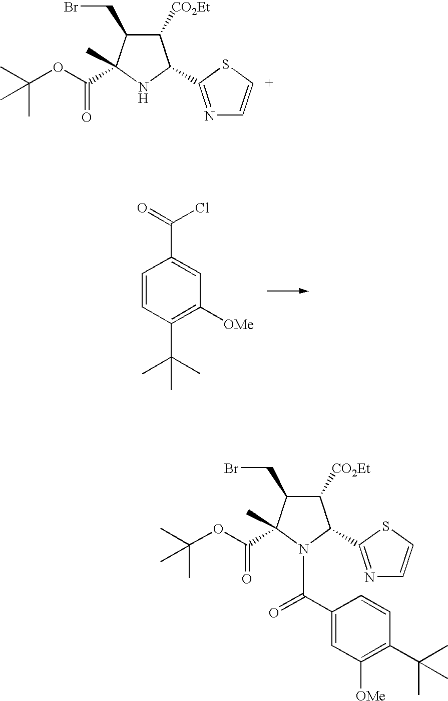

Step 1d. A mixture of the compound from step 1b (380 mg, 0.88 mmol), the compound from step 1c (348 mg, 4.0 mmol), and triethylamine (368 μL, 2.64 mmol) in methylene chloride (4 mL) is stirred at room temperature for 2 days. The reaction mixture is concentrated and purified by flash chromatography with EtOAc/Hexanes (0-40%) to give the desired compound as a pale yellow foam (330 mg, 60%).

ESIMS m/z=623.23 [M+H]⁺.

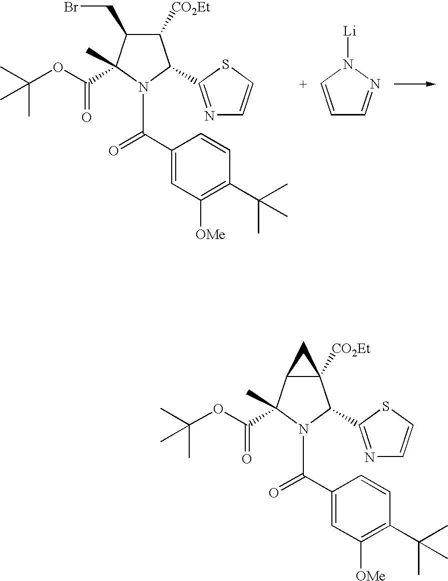

Step 1e. A solution of pyrazole (100 mg, 1.47 mmol) in freshly distilled THF (5 mL) is treated with n-BuLi (0.4 mL, 1 mmol, 2.5 M in hexanes) at −20° C. for 5 minutes. It is charged dropwisely to a solution of the compound from step 1d (50 mg, 0.08 mmol) in freshly distilled THF (10 mL). The resulted mixture is stirred for 2 h at room temperature and is quenched with saturated ammonium chloride and extracted with hexanes. The combined organics are washed with water and brine, dried (Na₂SO₄) and evaporated. The residue is purified by flash chromatography (silica, hexane-ethyl acetate) to give the title compound as a white powder (44 mg, 99%).

ESIMS m/z=543.43 [M+H]⁺.

### Example 2

### Compound of Formula (Ia) Wherein M=tert-butoxy, Q=4-tert-butyl-3-methoxybenzoyl, Z=1,3-thiazol-2-yl, X=—CH₂, Y=—CH₂OH, U=hydrogen, J=methyl

A solution of the compound from Example 1 (48 mg, 0.09 mmol) in THF (5 mL) is treated with lithium aluminum hydride (1M in THF, 0.5 mL, 0.5 mmol) at −45˜−35° C. for 100 min before being quenched with saturated ammonium chloride solution and extracted with hexanes. The combined organics are washed with water and brine, dried (Na₂SO₄), and evaporated. The residue is purified by flash chromatography (silica, hexane-ethyl acetate) to give the title compound as a white solid (19.1 mg, 42%).

ESIMS m/z=501.35 [M+H]⁺.

### Example 3

### Compound of Formula (Ia) Wherein M=tert-butoxy, Q=4-tert-butyl-3-methoxybenzoyl, Z=1,3-thiazol-2-yl, X=—CH₂, Y=—CH₂OCH₃, U=hydrogen, J=methyl

A mixture of the compound from Example 2 (4 mg, 0.008 mmol), NaOH (50% aqueous, 0.5 mL) and methyl iodide (0.5 mL) is stirred at room temperature for 8 h in the presence of tetrabutylammonium bromide (1 mg), before partition (ethyl acetate and water). The organics are washed with water and brine, dried (Na₂SO₄), and evaporated. The residue is purified by flash chromatography (silica, hexane-ethyl acetate) to give the title compound as a white solid (4 mg, 95%).

ESIMS m/z=515.39 [M+H]⁺.

### Example 4

### Compound of Formula (Ia) Wherein M=hydroxyl, Q=4-tert-butyl-3-methoxybenzoyl, Z=1,3-thiazol-2-yl, X=—CH₂, Y=—CH₂OCH₃, U=hydrogen, J=methyl

A solution of the compound from Example 3 (4 mg, 0.008 mmol) in trifluoroacetic acid (1 mL) is stirred at room temperature for 5 h before evaporation. The residue is purified by preparative TLC (hexane-ethyl acetate) to give the title compound as a white solid (2 mg, 57%).

ESIMS m/z=459.57 [M+H]⁺.

### Example 5

### Compound of Formula (Ia) Wherein M=hydroxyl, Q=4-tert-butyl-3-methoxybenzoyl, Z=1,3-thiazol-2-yl, X=—CH₂, Y=—CH₂OCOCF₃, U=hydrogen, J=methyl

A solution of the compound from Example 2 (5 mg, 0.01 mmol) in trifluoroacetic acid (0.5 mL) and methylene chloride (0.5 mL) is stirred at room temperature for 12 h before evaporation to give the crude title compound.

ESIMS m/z=541.22 [M+H]⁺.

### Example 6

### Compound of Formula (Ia) Wherein M=hydroxyl, Q=4-tert-butyl-3-methoxybenzoyl, Z=1,3-thiazol-2-yl, X=—CH₂, Y=—CH₂OH, U=hydrogen, J=methyl

A solution of the compound from Example 5 (4 mg, 0.01 mmol at most) in methanol (1 mL) is refluxed for 2 h before evaporation. The residue is purified by preparative TLC (hexane-ethyl acetate) to give the title compound as a white solid (2.9 mg).

ESIMS m/z=445.31 [M+H]⁺.

### Example 7

### Compound of Formula (Ia) Wherein M=hydroxy, Q=4-tert-butyl-3-methoxybenzoyl, Z=1,3-thiazol-2-yl, X=—CH₂, Y=—CH₂OCH₃, U=hydrogen, J=1H-pyrazol-1-ylmethyl

### Example 8

### Compound of Formula (Ia) Wherein M=hydroxy, Q=4-tert-butyl-3-methoxybenzoyl, Z=1,3-thiazol-2-yl, X=—CH₂, Y=—C(O)NH), U=hydrogen, J=1H-pyrazol-1-ylmethyl

### Example 9

### Compound of Formula (Ia) Wherein M=hydroxy, Q=4-tert-butyl-3-methoxybenzoyl, Z=1,3-thiazol-2-yl, X=—CH₂, Y=—C(O)NHBn, U=hydrogen, J=1H-pyrazol-1-ylmethyl

### Example 10

### Compound of Formula (Ia) Wherein M=hydroxy, Q=4-tert-butyl-3-methoxybenzoyl, Z=1,3-thiazol-2-yl, X=—CH₂, Y=—C(O)NHPh, U=hydrogen, J=1H-pyrazol-1-ylmethyl

### Example 11

### Compound of Formula (Ia) Wherein M=hydroxy, Q=4-tert-butyl-3-methoxybenzoyl, Z=1,3-thiazol-2-yl, X=—CH₂, Y=—CH₂OBn, U=hydrogen, J=1H-pyrazol-1-ylmethyl

### Example 12

### Compound of Formula (Ia) Wherein M=hydroxy, Q=4-tert-butyl-3-methoxybenzoyl, Z=1,3-thiazol-2-yl, X=—CH₂, Y=—SO₂Ph, U=hydrogen, J=1H-pyrazol-1-ylmethyl

### Example 13

### Compound of Formula (Ia) Wherein M=hydroxy, Q=4-tert-butyl-3-methoxybenzoyl, Z=1,3-thiazol-2-yl, X=—CH₂, Y=—NHMs, U=hydrogen, J=1H-pyrazol-1-ylmethyl

### Example 14

### Compound of Formula (Ia) Wherein M=hydroxy, Q=4-tert-butyl-3-methoxybenzoyl, Z=1,3-thiazol-2-yl, X=—CH₂, Y=—CO₂Et, U=hydrogen, J=1H-pyrazol-1-ylmethyl

### Example 15

### Compound of Formula (Ia) Wherein M=hydroxy, Q=4-tert-butyl-3-methoxybenzoyl, Z=1,3-thiazol-2-yl, X=—CH₂, Y=—CH₂OC(O)NH₂, U=hydrogen, J=1H-pyrazol-1-ylmethyl

### Example 16

### Compound of Formula (Ia) Wherein M=hydroxy, Q=4-tert-butyl-benzenesulfonyl, Z=1,3-thiazol-2-yl, X=—CH₂, Y=—CH₂OCH₃, U=hydrogen, J=1H-pyrazol-1-ylmethyl

### Example 17

### Compound of Formula (Ia) Wherein M=hydroxy, Q=4-tert-butyl-phenylcarbamoyl, Z=1,3-thiazol-2-yl, X=—CH₂, Y=—CH₂OCH₃, U=hydrogen, J=1H-pyrazol-1-ylmethyl

### Example 18

### Compound of Formula (Ia) Wherein M=hydroxy, Q=4-tert-butyl-3-bromobenzoyl, Z=1,3-thiazol-2-yl, X=—CH₂, Y=—CH₂OCH₃, U=hydrogen, J=1H-pyrazol-1-ylmethyl

### Example 19

### Compound of Formula (Ia) Wherein M=hydroxy, Q=4-tert-butyl-3-vinylbenzoyl, Z=1,3-thiazol-2-yl, X=—CH₂, Y=—CH₂OCH₃, U=hydrogen, J=1H-pyrazol-1-ylmethyl

### Example 20

### Compound of Formula (Ia) Wherein M=hydroxy, Q=2-fluoro-4-tert-butyl-5-vinylbenzoyl, Z=1,3-thiazol-2-yl, X=—CH₂, Y=—CH₂OCH₃, U=hydrogen, J=1H-pyrazol-1-ylmethyl

### Example 21

### Compound of Formula (Ia) Wherein M=hydroxy, Q=4-tert-butyl-3-methoxybenzoyl, Z=1,3,4-thiadiazol-2-yl, X=—CH₂, Y=—CH₂OCH₃, U=hydrogen, J=1H-pyrazol-1-ylmethyl

### Example 22

### Compound of Formula (Ia) Wherein M=hydroxy, Q=4-tert-butyl-3-methoxybenzoyl, Z=5-methylisoxazol-3-yl, X=—CH₂, Y=—CH₂OCH₃, U=hydrogen, J=1H-pyrazol-1-ylmethyl

### Example 23

### Compound of Formula (Ia) Wherein M=hydroxy, Q=4-tert-butyl-3-methoxybenzoyl, Z=thiophen-2-yl, X=—CH₂, Y=—CH₂OCH₃, U=hydrogen, J=1H-pyrazol-1-ylmethyl

### Example 24

### Compound of Formula (Ia) Wherein M=hydroxy, Q=4-tert-butyl-3-methoxybenzoyl, Z=thiophen-3-yl, X=—CH₂, Y=—CH₂OCH₃, U=hydrogen, J=1H-pyrazol-1-ylmethyl

### Example 25

### Compound of Formula (Ia) Wherein M=hydroxy, Q=4-tert-butyl-3-methoxybenzoyl, Z=furan-2-yl, X=—CH₂, Y=—CH₂OCH₃, U=hydrogen, J=1H-pyrazol-1-ylmethyl

### Example 26

### Compound of Formula (Ia) Wherein M=hydroxy, Q=4-tert-butyl-3-methoxybenzoyl, Z=furan-3-yl, X=—CH₂, Y=—CH₂OCH₃, U=hydrogen, J=1H-pyrazol-1-ylmethyl

### Example 27

### Compound of Formula (Ia) Wherein M=hydroxy, Q=4-tert-butyl-3-methoxybenzoyl, Z=1,3-oxazol-2-yl, X=—CH₂, Y=—CH₂OCH₃, U=hydrogen, J=1H-pyrazol-1-ylmethyl

### Example 28

### Compound of Formula (Ia) Wherein M=hydroxy, Q=4-tert-butyl-3-methoxybenzoyl, Z=phenyl, X=—CH₂, Y=—CH₂OCH₃, U=hydrogen, J=1H-pyrazol-1-ylmethyl

### Example 29

### Compound of Formula (Ia) Wherein M=hydroxy, Q=4-tert-butyl-3-methoxybenzoyl, Z=pyridin-2-yl, X=—CH₂, Y=—CH₂OCH₃, U=hydrogen, J=1H-pyrazol-1-ylmethyl

### Example 30

### Compound of Formula (Ia) Wherein M=hydroxy, Q=4-tert-butyl-3-methoxybenzoyl, Z=pyridin-3-yl, X=—CH₂, Y=—CH₂OCH₃, U=hydrogen, J=1H-pyrazol-1-ylmethyl

### Example 31

### Compound of Formula (Ia) Wherein M=hydroxy, Q=4-tert-butyl-3-methoxybenzoyl, Z=pyridin-4-yl, X=—CH₂, Y=—CH₂OCH₃, U=hydrogen, J=1H-pyrazol-1-ylmethyl

### Example 32

### Compound of Formula (Ia) Wherein M=hydroxy, Q=4-tert-butyl-3-methoxybenzoyl, Z=1,3-thiazol-2-yl, X=—CH₂, Y=—CH₂OCH₃, U=hydrogen, J=—CH═NOMe

### Example 33

### Compound of Formula (Ia) Wherein M=hydroxy, Q=4-tert-butyl-3-methoxybenzoyl, Z=1,3-thiazol-2-yl, X=—CH₂, Y=—CH₂OCH₃, U=hydrogen, J=—CH═NNMe₂

### Example 34

### Compound of Formula (Ia) Wherein M=hydroxy, Q=4-tert-butyl-3-methoxybenzoyl, Z=1,3-thiazol-2-yl, X=—CH₂, Y=—CH₂OCH₃, U=hydrogen, J=1,3-thiazol-4-ylmethyl

### Example 35

### Compound of Formula (Ia) Wherein M=hydroxy, Q=4-tert-butyl-3-methoxybenzoyl, Z=1,3-thiazol-2-yl, X=—CH₂, Y=—CH₂OCH₃, U=hydrogen, J=1,3-thiazol-2-ylmethyl

### Example 36

### Compound of Formula (Ia) Wherein M=hydroxy, Q=4-tert-butyl-3-methoxybenzoyl, Z=1,3-thiazol-2-yl, X=—CH₂, Y=—CH₂OCH₃, U=hydrogen, J=isoxazol-3-ylmethyl

### Example 37

### Compound of Formula (Ia) Wherein M=hydroxy, Q=4-tert-butyl-3-methoxybenzoyl, Z=1,3-thiazol-2-yl, X=—CH₂, Y=—CH₂OCH₃, U=hydrogen, J=pyridin-2-ylmethyl

### Example 38

### Compound of Formula (Ia) Wherein M=hydroxy, Q=4-tert-butyl-3-methoxybenzoyl, Z=1,3-thiazol-2-yl, X=—CH₂, Y=—CH₂OCH₃, U=hydrogen, J=—CH₂NMe₂

### Example 39

### Compound of Formula (Ia) Wherein M=hydroxy, Q=4-tert-butyl-3-methoxybenzoyl, Z=1,3-thiazol-2-yl, X=—CH₂, Y=—CH₂OCH₃, U=hydrogen, J=—CH₂CH₂NMe₂

### Example 40

### Compound of Formula (Ia) Wherein M=hydroxy, Q=4-tert-butyl-3-methoxybenzoyl, Z=1,3-thiazol-2-yl, X=—CH₂, Y=—CH₂OCH₃, U=hydrogen, J=—CH₂NHMs

### Example 41

### Compound of Formula (Ia) Wherein M=hydroxy, Q=4-tert-butyl-3-methoxybenzoyl, Z=1,3-thiazol-2-yl, X=—CH₂, Y=—CH₂OCH₃, U=hydrogen, J=—CH₂CH₂NHMs

### Example 42

### Compound of Formula (Ia) Wherein M=hydroxy, Q=4-tert-butyl-3-methoxybenzoyl, Z=1,3-thiazol-2-yl-methyl, X=—CH₂, Y=—CH₂OCH₃, U=hydrogen, J=1H-pyrazol-1-ylmethyl

### Example 43

### Compound of Formula (Ia) Wherein M=hydroxy, Q=4-tert-butyl-3-methoxybenzoyl, Z=isopropyl, X=—CH₂, Y=—CH₂OCH₃, U=hydrogen, J=1H-pyrazol-1-ylmethyl

### Example 44

### Compound of Formula (Ia) Wherein M=hydroxy, Q=4-tert-butyl-3-methoxybenzoyl, Z=1,3-thiazol-2-yl, X=CF₂, Y=—CH₂OCH₃, U=hydrogen, J=1H-pyrazol-1-ylmethyl

### Example 45

### Compound of Formula (Ia) Wherein M=hydroxy, Q=4-tert-butyl-3-methoxybenzoyl, Z=1,3-thiazol-2-yl, X=—CH₂CF₂—, Y=—CH₂OCH₃, U=hydrogen, J=1H-pyrazol-1-ylmethyl

### Example 46

### Compound of Formula (Ia) Wherein M=hydroxy, Q=4-tert-butyl-3-methoxybenzoyl, Z=1,3-thiazol-2-yl, X=—OCH₂CF₂—, Y=—CH₂OCH₃, U=hydrogen, J=1H-pyrazol-1-ylmethyl

While this invention has been particularly shown and described with references to preferred embodiments thereof, it will be understood by those skilled in the art that various changes in form and details may be made therein without departing from the scope of the invention encompassed by the appended claims.

What is claimed is:

1. A compound represented by Formula (I): 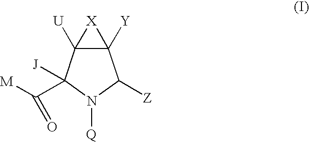
or a pharmaceutically acceptable salt, ester, stereoisomer, tautomer, solvate, prodrug, or combination thereof, wherein:
M at each occurrence is selected from the group consisting of:
a) —OR₁;
b) —NR₁R₂;
c) —SR₁; and
d) —R₁;
wherein R₁ and R₂ at each occurrence are each independently selected from the group consisting of:
1. hydrogen;
2. deuterium; and
3. —R₃;
wherein R₃ at each occurrence is selected from the group consisting of:
1)-C₁-C₈ alkyl, —C₂-C₈ alkenyl, —C₂-C₈ alkynyl or —C₃-C₈ cycloalkyl each containing 0, 1, 2, or 3 heteroatoms selected from O, S and N;
2) substituted —C₁-C₈ alkyl, substituted —C₂-C₈ alkenyl, substituted —C₂-C₈ alkynyl or substituted —C₃-C₈ cycloalkyl each containing 0, 1, 2, or 3 heteroatoms selected from O, S and N;
3) heterocyclic;
4) substituted heterocyclic;
5) aryl;
6) substituted aryl;
7) heteroaryl; and
8) substituted heteroaryl;
or R₁ and R₂ taken together with the nitrogen atom to which they are attached form a substituted or unsubstituted heterocyclic group;
Q at each occurrence is selected from the group consisting of:
a) —R₁;
b) —C(O)R₁;
c) —C(O)OR₁;
d) —C(O)NR₁R₂;
e) —S(O)ₙR₁, wherein n=0, 1, or 2;
f) —S(O)ₘNR₁R₂, m=1 or 2;
g) —(C═NR₄)NR₁R₂, wherein R₄ is independently R₁;
h) —P(O)R₁R₂;
i) —P(O)(OR₁)(OR₂);
j) —P(O)(NR₁R₂)(NR₂R₄); and
k) —P(O)(NR₁R₂)(OR₄);
U and Y at each occurrence is independently selected from the group consisting of:
a) halogen;
b) -M;
c) -Q;
d) —NO₂;
e) —CN;
f) —N₃;
g) —C(R₄)═N—O—R₁;
h) —C(R₄)═N—NR₁R₂;
i) —O-Q; and
j) —N(R₁)-Q;
Z at each occurrence is independently —R₁;
X is selected from the group consisting of: —C(A₁A₂)-, —C(A₁A₂)C(B₁B₂)-, or —W—C(A₁A₂)C(B₁B₂)-, wherein A₁, A₂, B₁, B₂ at each occurrence are each independently -M, halogen, or -Q; and W at each occurrence is O, S, or NR₁; and
J at each occurrence is selected from the group consisting of:
a) —C(R₄)═N—O—R₁;
b) —C(R₄)═N—NR₁R₂; and
c) —R₁.

2. A compound of claim 1 wherein M is hydroxy.

3. A compound of claim 1 wherein M is hydroxy and X is —C(A₁A₂)-.

4. A compound of claim 1 wherein M is hydroxy and X is —C(A₁A₂)C(B₁B₂)-.

5. A compound of claim 1 wherein M is hydroxy and X is —W—C(A₁A₂)C(B₁B₂)-.

6. A compound of claim 1 wherein Y is halogen, —OR₁, —NR₁R₂, or —C(R₄)═N—O—R₁.

7. A compound of claim 1 wherein J is —R₁, —C(R₄)═N—O—R₁ or —C(R₄)═N—NR₁R₂.

8. A compound of claim 1 wherein M is hydroxy, J is —R₁ or —C(R₄)═N—O—R₁.

9. A compound of claim 1 wherein Z is substituted or unsubstituted aryl or heteroaryl.

10. A compound of claim 1 wherein Q is —C(O)R₁.

11. A compound of claim 1 wherein M is hydroxy and Q is —C(O)R₁.

12. A compound according to claim 1 selected from the group consisting of:
Compound of Formula (Ia), wherein M=tert-butoxy, Q=4-tert-butyl-3-methoxybenzoyl, Z=1,3-thiazol-2-yl, X=—CH₂, Y=—CO₂Et, U=hydrogen, J=methyl;
Compound of Formula (Ia), wherein M=tert-butoxy, Q=4-tert-butyl-3-methoxybenzoyl, Z=1,3-thiazol-2-yl, X=—CH₂, Y=—CH₂OH, U=hydrogen, J=methyl;
Compound of Formula (Ia), wherein M=tert-butoxy, Q=4-tert-butyl-3-methoxybenzoyl, Z=1,3-thiazol-2-yl, X=—CH₂, Y=—CH₂OCH₃, U=hydrogen, J=methyl;
Compound of Formula (Ia), wherein M=hydroxyl, Q=4-tert-butyl-3-methoxybenzoyl, Z=1,3-thiazol-2-yl, X=—CH₂, Y=—CH₂OCH₃, U=hydrogen, J=methyl;
Compound of Formula (Ia), wherein M=hydroxyl, Q=4-tert-butyl-3-methoxybenzoyl, Z=1,3-thiazol-2-yl, X=—CH₂, Y=—CH₂OCOCF₃, U=hydrogen, J=methyl;
Compound of Formula (Ia), wherein M=hydroxyl, Q=4-tert-butyl-3-methoxybenzoyl, Z=1,3-thiazol-2-yl, X=—CH₂, Y=—CH₂OH, U=hydrogen, J=methyl;
Compound of Formula (Ia), wherein M=hydroxy, Q=4-tert-butyl-3-methoxybenzoyl, Z=1,3-thiazol-2-yl, X=—CH₂, Y=—CH₂OCH₃, U=hydrogen, J=1H-pyrazol-1-ylmethyl;
Compound of Formula (Ia), wherein M=hydroxy, Q=4-tert-butyl-3-methoxybenzoyl, Z=1,3-thiazol-2-yl, X=—CH₂, Y=—C(O)NH₂, U=hydrogen, J=1H-pyrazol-1-ylmethyl;
Compound of Formula (Ia), wherein M=hydroxy, Q=4-tert-butyl-3-methoxybenzoyl, Z=1,3-thiazol-2-yl, X=—CH₂, Y=—C(O)NHBn, U=hydrogen, J=1H-pyrazol-1-ylmethyl;
Compound of Formula (Ia), wherein M=hydroxy, Q=4-tert-butyl-3-methoxybenzoyl, Z=1,3-thiazol-2-yl, X=—CH₂, Y=—C(O)NHPh, U=hydrogen, J=1H-pyrazol-1-ylmethyl;
Compound of Formula (Ia), wherein M=hydroxy, Q=4-tert-butyl-3-methoxybenzoyl, Z=1,3-thiazol-2-yl, X=—CH₂, Y=—CH₂OBn, U=hydrogen, J=1H-pyrazol-1-ylmethyl;
Compound of Formula (Ia), wherein M=hydroxy, Q=4-tert-butyl-3-methoxybenzoyl, Z=1,3-thiazol-2-yl, X=—CH₂, Y=—SO₂Ph, U=hydrogen, J=1H-pyrazol-1-ylmethyl;
Compound of Formula (Ia), wherein M=hydroxy, Q=4-tert-butyl-3-methoxybenzoyl, Z=1,3-thiazol-2-yl, X=—CH₂, Y=—NHMs, U=hydrogen, J=1H-pyrazol-1-ylmethyl;
Compound of Formula (Ia), wherein M=hydroxy, Q=4-tert-butyl-3-methoxybenzoyl, Z=1,3-thiazol-2-yl, X=—CH₂, Y=—CO₂Et, U=hydrogen, J=1H-pyrazol-1-ylmethyl;
Compound of Formula (Ia), wherein M=hydroxy, Q=4-tert-butyl-3-methoxybenzoyl, Z=1,3-thiazol-2-yl, X=—CH₂, Y=—CH₂OC(O)NH₂, U=hydrogen, J=1H-pyrazol-1-ylmethyl;
Compound of Formula (Ia), wherein M=hydroxy, Q=4-tert-butyl-benzenesulfonyl, Z=1,3-thiazol-2-yl, X=—CH₂, Y=—CH₂OCH₃, U=hydrogen, J=1H-pyrazol-1-ylmethyl;
Compound of Formula (Ia), wherein M=hydroxy, Q=4-tert-butyl-phenylcarbamoyl, Z=1,3-thiazol-2-yl, X=—CH₂, Y=—CH₂OCH₃, U=hydrogen, J=1H-pyrazol-1-ylmethyl;
Compound of Formula (Ia), wherein M=hydroxy, Q=4-tert-butyl-3-bromobenzoyl, Z=1,3-thiazol-2-yl, X=—CH₂, Y=—CH₂OCH₃, U=hydrogen, J=1H-pyrazol-1-ylmethyl;
Compound of Formula (Ia), wherein M=hydroxy, Q=4-tert-butyl-3-vinylbenzoyl, Z=1,3-thiazol-2-yl, X=—CH₂, Y=—CH₂OCH₃, U=hydrogen, J=1H-pyrazol-1-ylmethyl;
Compound of Formula (Ia), wherein M=hydroxy, Q=2-fluoro-4-tert-butyl-5-vinylbenzoyl, Z=1,3-thiazol-2-yl, X=—CH₂, Y=—CH₂OCH₃, U=hydrogen, J=1H-pyrazol-1-ylmethyl;
Compound of Formula (Ia), wherein M=hydroxy, Q=4-tert-butyl-3-methoxybenzoyl, Z=1,3,4-thiadiazol-2-yl, X=—CH₂, Y=—CH₂OCH₃, U=hydrogen, J=1H-pyrazol-1-ylmethyl;
Compound of Formula (Ia), wherein M=hydroxy, Q=4-tert-butyl-3-methoxybenzoyl, Z=5-methylisoxazol-3-yl, X=—CH₂, Y=—CH₂OCH₃, U=hydrogen, J=1H-pyrazol-1-ylmethyl;
Compound of Formula (Ia), wherein M=hydroxy, Q=4-tert-butyl-3-methoxybenzoyl, Z=thiophen-2-yl, X=—CH₂, Y=—CH₂OCH₃, U=hydrogen, J=1H-pyrazol-1-ylmethyl;
Compound of Formula (Ia), wherein M=hydroxy, Q=4-tert-butyl-3-methoxybenzoyl, Z=thiophen-3-yl, X=—CH₂, Y=—CH₂OCH₃, U=hydrogen, J=1H-pyrazol-1-ylmethyl;
Compound of Formula (Ia), wherein M=hydroxy, Q=4-tert-butyl-3-methoxybenzoyl, Z=furan-2-yl, X=—CH₂, Y=—CH₂OCH₃, U=hydrogen, J=1H-pyrazol-1-ylmethyl;
Compound of Formula (Ia), wherein M=hydroxy, Q=4-tert-butyl-3-methoxybenzoyl, Z=furan-3-yl, X=—CH₂, Y=—CH₂OCH₃, U=hydrogen, J=1H-pyrazol-1-ylmethyl;
Compound of Formula (Ia), wherein M=hydroxy, Q=4-tert-butyl-3-methoxybenzoyl, Z=1,3-oxazol-2-yl, X=—CH₂, Y=—CH₂OCH₃, U=hydrogen, J=1H-pyrazol-1-ylmethyl;
Compound of Formula (Ia), wherein M=hydroxy, Q=4-tert-butyl-3-methoxybenzoyl, Z=phenyl, X=—CH₂, Y=—CH₂OCH₃, U=hydrogen, J=1H-pyrazol-1-ylmethyl;
Compound of Formula (Ia), wherein M=hydroxy, Q=4-tert-butyl-3-methoxybenzoyl, Z=pyridin-2-yl, X=—CH₂, Y=—CH₂OCH₃, U=hydrogen, J=1H-pyrazol-1-ylmethyl;
Compound of Formula (Ia), wherein M=hydroxy, Q=4-tert-butyl-3-methoxybenzoyl, Z=pyridin-3-yl, X=—CH₂, Y=—CH₂OCH₃, U=hydrogen, J=1H-pyrazol-1-ylmethyl;
Compound of Formula (Ia), wherein M=hydroxy, Q=4-tert-butyl-3-methoxybenzoyl, Z=pyridin-4-yl, X=—CH₂, Y=—CH₂OCH₃, U=hydrogen, J=1H-pyrazol-1-ylmethyl;
Compound of Formula (Ia), wherein M=hydroxy, Q=4-tert-butyl-3-methoxybenzoyl, Z=1,3-thiazol-2-yl, X=—CH₂, Y=—CH₂OCH₃, U=hydrogen, J=—CH═NOMe;
Compound of Formula (Ia), wherein M=hydroxy, Q=4-tert-butyl-3-methoxybenzoyl, Z=1,3-thiazol-2-yl, X=—CH₂, Y=—CH₂OCH₃, U=hydrogen, J=—CH═NNMe₂;
Compound of Formula (Ia), wherein M=hydroxy, Q=4-tert-butyl-3-methoxybenzoyl, Z=1,3-thiazol-2-yl, X=—CH₂, Y=—CH₂OCH₃, U=hydrogen, J=1,3-thiazol-4-ylmethyl;
Compound of Formula (Ia), wherein M=hydroxy, Q=4-tert-butyl-3-methoxybenzoyl, Z=1,3-thiazol-2-yl, X=—CH₂, Y=—CH₂OCH₃, U=hydrogen, J=1,3-thiazol-2-ylmethyl;
Compound of Formula (Ia), wherein M=hydroxy, Q=4-tert-butyl-3-methoxybenzoyl, Z=1,3-thiazol-2-yl, X=—CH₂, Y=—CH₂OCH₃, U=hydrogen, J=isoxazol-3-ylmethyl;
Compound of Formula (Ia), wherein M=hydroxy, Q=4-tert-butyl-3-methoxybenzoyl, Z=1,3-thiazol-2-yl, X=—CH₂, Y=—CH₂OCH₃, U=hydrogen, J=pyridin-2-ylmethyl;
Compound of Formula (Ia), wherein M=hydroxy, Q=4-tert-butyl-3-methoxybenzoyl, Z=1,3-thiazol-2-yl, X=—CH₂, Y=—CH₂OCH₃, U=hydrogen, J=—CH₂NMe₂;
Compound of Formula (Ia), wherein M=hydroxy, Q=4-tert-butyl-3-methoxybenzoyl, Z=1,3-thiazol-2-yl, X=—CH₂, Y=—CH₂OCH₃, U=hydrogen, J=—CH₂CH₂NMe₂;
Compound of Formula (Ia), wherein M=hydroxy, Q=4-tert-butyl-3-methoxybenzoyl, Z=1,3-thiazol-2-yl, X=—CH₂, Y=—CH₂OCH₃, U=hydrogen, J=—CH₂NHMs;
Compound of Formula (Ia), wherein M=hydroxy, Q=4-tert-butyl-3-methoxybenzoyl, Z=1,3-thiazol-2-yl, X=—CH₂, Y=—CH₂OCH₃, U=hydrogen, J=—CH₂CH₂NHMs;
Compound of Formula (Ia), wherein M=hydroxy, Q=4-tert-butyl-3-methoxybenzoyl, Z=1,3-thiazol-2-yl-methyl, X=—CH₂, Y=—CH₂OCH₃, U=hydrogen, J=1H-pyrazol-1-ylmethyl;
Compound of Formula (Ia), wherein M=hydroxy, Q=4-tert-butyl-3-methoxybenzoyl, Z=isopropyl, X=—CH₂, Y=—CH₂OCH₃, U=hydrogen, J=1H-pyrazol-1-ylmethyl;
Compound of Formula (Ia), wherein M=hydroxy, Q=4-tert-butyl-3-methoxybenzoyl, Z=1,3-thiazol-2-yl, X=CF₂, Y=—CH₂OCH₃, U=hydrogen, J=1H-pyrazol-1-ylmethyl;
Compound of Formula (Ia), wherein M=hydroxy, Q=4-tert-butyl-3-methoxybenzoyl, Z=1,3-thiazol-2-yl, X=—CH₂CF₂—, Y=—CH₂OCH₃, U=hydrogen, J=1H-pyrazol-1-ylmethyl;
Compound of Formula (Ia), wherein M=hydroxy, Q=4-tert-butyl-3-methoxybenzoyl, Z=1,3-thiazol-2-yl, X=—OCH₂CF₂—, Y=—CH₂OCH₃, U=hydrogen, J=1H-pyrazol-1-ylmethyl.

13. A pharmaceutical composition comprising a compound or a combination of compounds according to claim 1 or a pharmaceutically acceptable salt, stereoisomer, tautomer, prodrug, salt of a prodrug, or combination thereof, in combination with a pharmaceutically acceptable carrier or excipient.

14. A method of inhibiting the replication of an RNA-containing virus comprising contacting said virus with a therapeutically effective amount of a compound or combination of compounds of claim 1, or a pharmaceutically acceptable salt, stereoisomer, tautomer, prodrug, salt of a prodrug, or combination thereof.

15. A method of treating or preventing infection caused by an RNA-containing virus comprising administering to a patient in need of such treatment a therapeutically effective amount of a compound or combination of compounds of claim 1, or a pharmaceutically acceptable salt, stereoisomer, tautomer, prodrug, salt of a prodrug, or combination thereof.

16. The method of claim 15 wherein the RNA-containing virus is hepatitis C virus.

17. The method of claim 15 further comprising the step of co-administering one or more agents selected from the group consisting of a host immune modulator and a second antiviral agent, or a combination thereof.

18. The method of claim 17 wherein the host immune modulator is selected from the group consisting of interferon-alpha, pegylated-interferon-alpha, interferon-beta, interferon-gamma, a cytokine, a vaccine and a vaccine comprising an antigen and an adjuvant.

19. The method of claim 17 wherein the second antiviral agent inhibits replication of HCV by inhibiting host cellular functions associated with viral replication.

20. The method of claim 17 wherein the second antiviral agent inhibits the replication of HCV by targeting proteins of the viral genome.

21. The method of claim 20 wherein said targeting protein is selected from the group consisting of helicase, protease, polymerase, metal loprotease, and IRES.

22. The method of claim 15 further comprising the step of co-administering an agent or combination of agents that treat or alleviate symptoms of HCV infection including cirrhosis and inflammation of the liver.

23. The method of claim 15 further comprising the step of co-administering one or more agents that treat patients for disease caused by hepatitis B (HBV) infection.

24. The method of claim 15 further comprising the step of co-administering one or more agents that treat patients for disease caused by human immunodeficiency virus (HIV) infection.

25. A process comprising the steps of treating compound of the following formula: 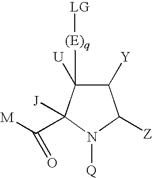
wherein E is a carbon or heteroatom centered moiety; and q is an integer from 1 to 3, and LG is a leaving group as defined previously, with a base which can be added externally or generated from the LG-group internally, optionally in the presence of an alkylating reagent, and optionally in the presence of a transitional metal catalyst and a ligand in an aprotic solvent to produce 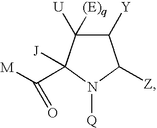
wherein E₂ is a carbon or heteroatom centered moiety and M, Z, Y, Q, U and J are as defined in claim 1.
